(19) United States
(12) Patent Application Publication
(10) Pub. No.: US 2004/0171019 A1
(43) Pub. Date: Sep. 2, 2004

(54) PIN1 peptidyl-prolyl isomerase polypeptides, their crystal structures, and use thereof for drug design

(75) Inventors: David Matthews, Encinitas, CA (US); Eleanor Dagostino, San Diego, CA (US); Rose Ann Ferre, Carlsbad, CA (US); Smita Gaur, San Diego, CA (US); Chuangxing Guo, San Diego, CA (US); Xinjun Hou, San Diego, CA (US); Stephen Margosiak, Escondido, CA (US); Barbara Mroczkowski, Encinitas, CA (US); Grace Reiko Nakayama, San Diego, CA (US); Hans Parge, San Diego, CA (US); Jeff Zhu, San Diego, CA (US)

Correspondence Address: AGOURON PHARMACEUTICALS, INC., 10350 NORTH TORREY PINES ROAD, LA JOLLA, CA, 92037, US

(73) Assignee: Agouron Pharmaceuticals, Inc.

(21) Appl. No.: 10/616,003

(22) Filed: Dec. 19, 2003

Related U.S. Application Data
(60) Provisional application No. 60/394,889, filed on Jul. 9, 2002.

(51) Int. Cl. C12Q 1/68; C07H 21/04; C12N 9/64

(52) U.S. Cl. 435/6; 435/69.1; 435/320.1; 435/325; 435/226; 536/23.2

(57) ABSTRACT

Polypeptides containing the PIN1 peptidyl-prolyl isomerase domain but not containing the PIN1 WW domain are described. Also described are crystal structures of these polypeptides, including the crystal structure of a PIN1 PPIase:ligand complex. The structure coordinate data derived from these crystals provides a three-dimensional description of the substrate-binding site of PIN1 peptidyl-prolyl isomerase useful in drug discovery and design for the identification and design of modulators of PIN1 peptidyl-prolyl isomerase activity.

This application claims priority under 35 USC § 119 to U.S. Provisional Application No. 60/394,889.

### FIELD AND INDUSTRIAL APPLICABILITY OF THE INVENTION

The present invention relates to mutant PIN1 polypeptides that lack a PIN1 WW domain and the polynucleotides that encode them. The invention also relates to the X-ray crystal structures of theses polypeptides. Additionally, the invention relates to crystallized complexes of the mutant PIN1 PPIase polypeptides and small entities that bind to the PIN1 PPIase substrate-binding domain. The invention also relates to the use of the atomic coordinates determined from such crystal structures for the use in drug design and development.

### BACKGROUND OF THE INVENTION

The cell cycle represents a series of ordered processes that ultimately results in the duplication of a cell. Somatic cell division consists of two sequential processes, mainly DNA replication followed by chromosomal separation. The cell spends most of its time preparing for these events in a growth cycle (interphase), which in turn consists of three subphases: initial gap (G₁), synthesis (S), and secondary gap (G₂). In G₁, the cell undergoes a high rate of biosynthesis. The S phase begins when DNA synthesis starts and ends when the DNA content of the nucleus has doubled. The cell then enters G₂, which lasts until the cell enters the final phase of division, mitosis (M). The M phase begins with nuclear envelope breakdown, chromosome condensation and formation of two identical sets of chromosomes that are separated into two new nuclei. This is followed by cell division (cytokineis), which results in two daughter cells. This separation terminates the M phase and marks the beginning of interphase for the new cells.

Entry into mitosis is a highly regulated event in normal cells. In eukaryotic cells studied to date, Cdc2/cyclin B, a Ser/Thr kinase, regulates entry into mitosis (Nurse, Nature 344:503-508 (1990)). To prevent inappropriate mitotic activity, the activity of Cdc2/cyclin B is tightly regulated. The Cdc/cyclin complex is both positively and negatively regulated by phosphorylation. Cdc2/cyclin B, when activated by dephosphorylation by Cdc25, drives cells into mitosis.

One regulator of Cdc25 is PIN1, a peptidyl-prolyl isomerase (PPIase). PIN1 is a member of the parvulin family of PPIases and catalyzes rotation about the peptide bond preceding a proline residue. This reaction is suggested to be important in the folding and trafficking of some proteins (Schmid, Curr. Biol. 5:993-994 (1995)). Other well-characterized PPIase families include the cyclophilins, and the FK506-binding proteins (FKBPs), which are targets of the immunosuppresive drugs cyclosporin A and FK506, respectively. Parvulins, such as PIN1, the cyclophilins, and the FKBPs are unrelated in primary sequence.

PIN1 has been identified in all eukaryotic organisms where examined, including plants, yeast, insects and mammals (Hanes et al., Yeast 5:55-72 (1989); Lu et al., Nature 380:544-547 (1996); Maleszka et al., Proc. Natl. Acad. Sci. U.S.A. 93:447-451 (1996)). The yeast (Ess1) and Drosophila (dodo) PIN1 orthologues have high identity to human-expressed sequence tags, which ultimately led to the cloning of the human dodo gene called PIN1 (Maleszka et al., Gene 203:89-93 (1997)). The fly dodo gene is reported to be 45% identical to the yeast gene, Essl.

Using a yeast two-hybrid screen of a human cDNA library, human PIN1 was originally identified as a binding protein of the fungi Aspergillus nidulens protein NIMA, (Lu et al., 1996, supra). NIMA is a kinase that drives cells into mitosis and is reported to be negatively regulated by PIN1. Depletion of NIMA in A. nidulans cells is reported to lead to cell cycle arrest in G₂, while overexpression is reported to promote premature mitosis. Ser/Thr kinase Cdc2/cyclin B may be the analogous NIMA kinase in human cells, although another NIMA-like pathway in human cells is postulated to exist (Lu et al., Cell 81:413-424 (1995)).

Modulation of PIN1 activity is reported to result in dramatic morphological cellular phenotypes. For example, overexpression of PIN1 in Hela cells was reported to cause a G₂ arrest while depletion caused mitotic arrest, the opposite phenotypes observed with NIMA modulation (Lu et al., 1996, supra; Crenshaw et al., EMBO J. 17:1315-1327 (1998)). Additionally, decreasing PIN1 protein expression by full-length antisense expression has been reported to cause cells to progress into mitosis prematurely, to contain aberrant nuclei due to premature chromosome condensation and to induce apoptosis (Lu et al., 1996, supra). These data indicate that PIN1 is a negative regulator of mitosis through interactions with a mammalian functional homologue of NIMA and is required for progression through mitosis. Further, depletion of PIN1 is also postulated to play a role Alzheimer's disease (Lu et al., Nature 399:784-788 (1999)).

In vitro, PIN1 has been reported to interact with mitotic proteins also recognized by the MPM-2 antibody (Crenshaw et al., supra; Lu et al., Science 283:1325-1328 (1999); Ranganathan et al., Cell 89:875-886 (1997); and Yaffe et al., Science 278:1957-1960 (1997)). The MPM-2 monoclonal antibody recognizes a phospho-Ser/Thr-Pro epitope on about approximately 50 proteins associated with mitosis, including important mitotic regulators, such as Cdc25, Wee1, Cdc27, Map 4, and NIMA (Davis et al., Proc. Natl. Acad. Sci. U.S.A. 80:2926-2930 (1983); Kuang et al., Proc. Natl. Acad. Sci. U.S.A. 86:4982-4986 (1989); Westendorf et al., Proc. Natl. Acad. Sci. U.S.A. 91:714-718 (1994); and Stuckenberg et al., Curr Biol. 7:338-348 (1997)). PIN1 has also been reported to interact with important upstream regulators of Cdc2/cyclin B including Cdc25 and its known regulator, P1×1 (Shen et al., Genes Dev. 12:706 (1998); Crenshaw et al., EMBO J. 17:1315-1327 (1998)). PIN1, due to its enzymatic action may remove Cdc25 and P1×1 from play by causing their degradation within the cell.

Studies indicate that the biological function of PIN1 depends on a functional PPIase active site (Lu et al., 1999, supra). Studies also indicate that PIN1 recognizes its substrates (mitosis-specific phosphoproteins) through its WW domain. The WW domain is a protein recognition motif that is prevalent throughout biology. However, the PIN1 WW domain is unique in that it requires its ligand protein to contain a phosphorylated serine. As with the PPIase domain, a functional WW domain is reported to be essential for biological function of PIN1. This is consistent with the model where PIN1 recognizes its substrates through the WW domain followed by completion of its essential catalytic role.

Full-length PIN1 protein and the nucleotide sequence encoding full-length PIN1 are disclosed in U.S. Pat. Nos. 5,952,467 and 5,972,697. Sequence information for PIN1 amino-acid sequence and mRNA sequence have been deposited in GenBank under accession numbers NM006221 (mRNA) and S68520 (protein). The mRNA sequence for dodo is deposited in GenBank under accession number U35140. Mouse PIN1 mRNA sequence is deposited in GenBank under accession number NM_{—}023371.

Ranganathan, et al., (Cell, 89: 875-886 (1997); International Publication No. WO 99/63931; and U.S. patent application Publication No. US2001/0016346 A1) present the crystal structure of full-length PIN1 reportedly complexed with an AlaPro dipeptide. The atomic coordinates for the crystal structure reported by Ranganathan et al. are available in the Protein Data Bank (PDB). Information from the PDB internet site (http://www.rcsb.org/pdp/) indicates that this data was deposited on Jun. 21, 1998, and released on Oct. 14, 1998.

Neoplastic cells, due to their inherent genetic instability, have lost many of the control mechanisms regulating cell division. Such neoplastic cells are more susceptible to cell-cycle modulation or intervention as a means of inducing cell death by apoptosis. Further, because alterations in cell-cycle control are one of the differences between normal cells and cancer cells, proteins involved in cell-cycle control are attractive targets for developing cytotoxic agents effective for use in cell proliferative disorders. One such target is PIN1.

PIN1 inhibitors will be cytotoxic to cells and affect cells in the G₂ phase of the cell cycle. Transformed cells will be hypersensitive to a PIN1 inhibitor due to their genomic instability and decreased and inefficient regulation of the cell cycle.

Inhibitors of PIN1 have been described in the literature. For example, Hennig et al., (Biochemistry 37: 5953-5960 (1998)) report that juglone (5-hydroxy-1,4-naphthoquinone) selectively inhibits several parvulins, including human PIN1. Noel et al. (International Publication No. WO 99/63931 and U.S. patent application Publication No. US201/0016346 A1), using data based on the crystal structure derived from full-length human PIN1, describe certain compounds as being inhibitors of PIN1. Lu et al. (International Publication No. PCT WO 99/12962) report inhibitors that mimic the phospho-Ser/Thr moiety of the phosphoserine or phosphothreonine-proline peptidyl prolyl isomerase substrate.

Because of the important role that PIN1 plays in the regulation of the cell cycle, stable recombinant polypeptides containing the PIN1 PPIase binding domain that are capable of manipulation for biochemical assays and crystallography studies are needed for the development of compounds that are modulators of PIN1 PPIase activity.

### SUMMARY OF THE INVENTION

The present invention relates to polynucleotides and the polypeptides they encode. These polynucleotides encode for the PIN1 PPIase domain but do not encode for the PIN1 WW domain. The genetically engineered polypeptides encoded by the polynucleotides described herein may also contain discreet amino acid substitutions as compared to the wild-type PIN1 PPIase domain. The polypeptides described herein are advantageous over full-length wild-type PIN1 because they have better crystallization properties when crystallized with ligands that interact with the PPIase substrate-binding domain.

One embodiment of the invention includes polynucleotides that encode for a PIN1 peptidyl-prolyl isomerase (PPIase) polypeptide that is devoid of the WW domain.

A preferred embodiment is an isolated polynucleotide that encodes a polypeptide including the amino acid sequence of SEQ ID NO:2 and which does not have sequences that encode for a WW domain.

Preferred is an isolated polynucleotide including the polynucleotide sequence of SEQ ID NO:1 where the polynucleotide does not have sequences that encode for a WW domain.

Another preferred polynucleotide is an isolated polynucleotide that encodes a polypeptide including the amino acid sequence of SEQ ID NO:4 and which does not have sequences that encode for a WW domain.

Yet another preferred polynucleotide is an isolated polynucleotide including the polynucleotide sequence of SEQ ID NO:3 where the polynucleotide does not have sequences that encode for a WW domain.

In a preferred embodiment, the polynucleotides described herein encode for at least one proteolytic cleavage site. A preferred cleavage site is a thrombin cleavage site.

In yet another preferred embodiment, the polynucleotides described herein include at least one sequence that encodes a histidine tag.

The invention also relates to the isolated polypeptides encoded by the polynucleotides described herein. These polypeptides contain a PIN1 PPIase domain but not a WW domain. Preferred polypeptides include the isolated polypeptides having the amino acid sequences of SEQ ID NO:2 or SEQ ID NO:4.

Another embodiment of the invention is a vector that includes at least one of the isolated polynucleotides described herein. A preferred vector includes a polynucleotide that encodes for a PIN1 PPIase but does not have sequences that encode for a WW domain.

In a preferred embodiment, the vector is an expression vector that includes one of the polynucleotides described herein operably linked to a promoter. A preferred polynucleotide for expression is one that encodes for a PIN1 PPIase but does not have sequences that encode for a WW domain.

The invention also relates' to a eukaryotic cell line or prokaryotic cell transformed or transfected with a vector that includes one of the polynucleotides described herein. Preferably the eukaryotic cell line or prokaryotic cell is transformed or transfected with a vector that includes a polynucleotide that encodes for a PIN1 PPIase but does not have sequences that encode for a WW domain.

Another embodiment of the invention is a method of producing a PIN1 PPIase polypeptide where the method includes the following steps: (a) culturing a eukaryotic cell line or prokaryotic cell that has been transformed or transfected with a polynucleotide that encodes for a PIN1 PPIase and which does not have sequences that encode for a WW domain under conditions such that the polypeptide is expressed; and (b) recovering the polypeptide.

The invention also relates to a method of assaying a compound for its PIN1 modulating ability. The method includes the following steps: adding a test compound to a polypeptide comprising a PIN1 peptidyl-prolyl isomerase wherein the polypeptide does not contain a WW domain; measuring the polypeptide's peptidyl-prolyl isomerase activity; and determining if the activity of the polypeptide is modulated by the test compound.

A preferred method for assaying a compound for its PIN1 modulating ability is a high-throughput assay that includes the following steps: in a multiple vessel format, such as microwell plate, test compounds are added to a polypeptide comprising a PIN1 peptidyl-prolyl isomerase wherein the polypeptide does not contain a WW domain; measuring the polypeptide's peptidyl-prolyl isomerase activity; and determining if the activity of the polypeptide is modulated by the test compounds screened.

Still another embodiment of the invention is a crystal structure of a PIN1 PPIase polypeptide that is devoid of the WW domain. Preferred are crystal structures of the polypeptides having the amino acid sequence of SEQ ID NO:2, SEQ ID.NO:4, or fragments thereof.

In a preferred embodiment the crystal structures diffract X-rays at a resolution value greater than or equal to 3 Å. In a more preferred embodiment, the crystal structures diffract X-rays at a resolution value of greater than or equal to 2 Å.

In another preferred embodiment, the crystal structure of the PIN1 PPIase crystal structure has a three-dimensional structure characterized by the structure coordinates of Table II.

Another embodiment of the invention is a crystal structure of a PIN1 PPIase polypeptide:ligand complex, wherein the polypeptide does not contain a WW domain. Preferably the polypeptide in the complex includes the amino acid sequence of SEQ ID NO:2 or SEQ ID NO:4.

In a preferred embodiment, the crystal of the PIN1 PPIase polypeptide:ligand complex diffracts X-rays at a resolution of greater than or equal to 3.0 Å. In a more preferred embodiment, the crystal structure diffracts X-rays at a resolution of greater than or equal to 2 Å.

In another preferred embodiment, the ligand in the PIN1 PPIase polypeptide:ligand complex is a modulator of PIN1 peptidyl-prolyl isomerase activity.

A preferred modulator has the following formula:

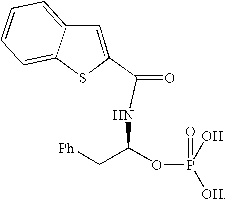

Another embodiment of the invention is a PIN1 PPIase polypeptide:ligand complex crystal structure having a three-dimensional structure characterized by the structure coordinates of Table III.

The invention also relates to a method of using the three-dimensional structure of the PIN1 PPIase polypeptide:compound I complex as defined by the structure coordinates of Table III or a portion thereof in a drug discovery strategy including the following steps:

(a) selecting a potential drug by using computer-aided drug design with the three-dimensional structure determined from one or more sets of atomic coordinates in Table III, wherein the selecting is performed in conjunction with computer modeling;

(b) contacting the potential drug with a polypeptide containing a functional PIN1 peptidyl-prolyl isomerase; and

(c) detecting the binding of the potential drug with the polypeptide, wherein a potential drug is selected for further analysis if the potential drug binds to the polypeptide.

Another preferred method described herein uses the three-dimensional structure of the PIN1 PPIase polypeptide:compound I complex as defined by the structure coordinates of Table III, or a portion thereof, in a drug discovery strategy that includes the following steps:

(a) selecting a potential drug by using computer-aided drug design with the three-dimensional structure determined from one or more sets of structure coordinates in Table III, wherein the selecting is performed in conjunction with computer modeling;

(b) contacting the potential drug with a polypeptide containing a functional PIN1 peptidyl-prolyl isomerase; and

(c) determining if the potential drug modulates the peptidyl-prolyl isomerase activity of a polypeptide containing a PIN1 peptidyl-prolyl isomerase.

Also described is a method for evaluating the potential of a chemical entity to associate with a molecule or molecular complex including a binding pocket defined by structure coordinates of PIN1 PPIase amino acids His59, Leu61, Lys63, Ser67, Arg68, Arg69, Cysi 13, Leu122, Met130, Gln131, Phe134, Glu135, Thr152, Ser154, and His157, according to Table III, including the steps of:

(a) employing computational means to perform a fitting operation between the chemical entity and a binding pocket defined by structure coordinates of PIN1 PPIase amino acids His59, Leu61, Lys63, Ser67, Arg68, Arg69, Cys113, Leu122, Met130, Gln131, Phe134, Glu135, Thr152, Ser154, and His157, according to Table III; and

(b) analyzing the results of the fitting operation to quantify the association between the chemical entity and the binding pocket.

A method is described for evaluating the potential of a chemical entity to associate with a molecule or molecular complex including a binding pocket defined by structure coordinates of PIN1 PPIase amino acids Arg54, Arg56, His59, Leu61, Lys63, Ser67, Arg68, Arg69, Ser72, Trp73, Ser111, Asp112, Cys113, Ser114, Ser115, Ala116, Lys117, Ala118, Arg119, Gly120, Asp121, Leu122, Gly123, Ala124, Phe125, Ser126, Arg127, Gly128, Gln129, Met130, Gln131, Lys132, Pro133, Phe134, Glu135, Thr152, Asp153, Ser154, and His157 according to Table III, including the steps of:

(a) employing computational means to perform a fitting operation between the chemical entity and a binding pocket defined by the structure coordinates of PIN1 PPIase amino acids Arg54, Arg56, His59, Leu61, Lys63, Ser67, Arg68, Arg69, Ser72, Trp73, Ser111, Asp112, Cys113, Ser114, Ser115, Ala116, Lys117, Ala118, Arg119, Gly120, Asp121, Leu122, Gly123, Ala124, Phe125, Ser126, Arg127, Gly128, Gln129, Met130, Gln131, Lys132, Pro33, Phe134, Glu135, Thr152, Asp153, Serl54, and His157 according to Table III; and

(b) analyzing the results of the fitting operation to quantify the association between the chemical entity and the binding pocket.

Also described herein is a method for identifying a modulator of a molecule including a PIN1 PPIase substrate-binding domain including the steps of:

(a) using the structure coordinates of PIN1 PPIase amino acids His59, Leu61, Lys63, Ser67, Arg68, Arg69, Cys113, Leu122, Met130, Gln131, Phe134, Glu135, Thr152, Ser154, and His157, according to Table III to generate a three-dimensional structure of molecule including a PIN1 PPIase or PPIase-like substrate-binding pocket;

(b) employing the three-dimensional structure to design or select the modulator;

(c) synthesizing or obtaining the modulator; and

(d) contacting the modulator with the molecule to determine the ability of the modulator to interact with the molecule.

Another method described for identifying a modulator of a molecule including a PIN1 PPIase substrate-binding domain includes the steps of:

(a) using the structure coordinates of PIN PPIase amino acids Arg54, Arg56, His59, Leu61, Lys63, Ser67, Arg68, Arg69, Ser72, Trp73, Ser111, Asp112, Cys113, Ser114, Ser115, Ala116, Lys117, Ala118, Arg119, Gly120, Asp121, Leu122, Gly123, Ala124, Phe125, Ser126, Arg127, Gly128, Gln129, Met130, Gln131, Lys132, Pro133, Phe134, Glu135, Thr152, Asp153, Ser154, and His157 according to Table III to generate a three-dimensional structure of the molecule including a PIN1 PPIase or PPIase-like substrate-binding pocket;

(b) employing the three-dimensional structure to design or select the modulator;

(c) synthesizing or obtaining the modulator; and

(d) contacting the modulator with the molecule to determine the ability of the modulator to interact with the molecule.

Yet another method for identifying a modulator of a molecule including a PIN1 PPIase substrate-binding domain includes the steps of:

(a) using the structure coordinates of all the amino acids of PIN1 PPIase according to Table III to generate a three-dimensional structure of the molecule including a PIN1 PPIase or PPIase-like substrate-binding pocket;

(b) employing the three-dimensional structure to design or select the modulator;

(c) synthesizing or obtaining the modulator; and

(d) contacting the modulator with the molecule to determine the ability of the modulator to interact with the molecule.

A preferred embodiment of the invention is a machine-readable medium having stored thereon data including the structure coordinates of a PIN1 PPIase substrate-binding site amino acids His59, Leu61, Lys63, Ser67, Arg68, Arg69, Cys113, Leu122, Met130, Gln131, Phe134, Glu135, Thr152, Ser154, and His157 according to Table III.

Another preferred embodiment is a machine-readable medium having stored thereon data including the structure coordinates of a PIN1 PPIase substrate-binding site amino acids Arg54, Arg56, His59, Leu61, Lys63, Ser67, Arg68, Arg69, Ser72, Trp73, Ser111, Asp112, Cys113, Ser114, Ser115, Ala116, Lys117, Ala118, Arg119, Gly120, Asp121, Leu122, Gly123, Ala124, Phe125, Ser126, Arg127, Gly128, Gln129, Met130, Gln131, Lys132, Pro133, Phe134, Glu135, Thr152, Asp153, Ser154, and His157 according to Table III.

Yet another preferred embodiment is a machine-readable medium having stored thereon data including all the structure coordinates of a PIN1 PPIase:Compound I complex according to Table III.

The invention also describes a method of obtaining structural information about a molecule or a molecular complex of unknown structure by using the structure coordinates set forth in Table III, including the steps of:

(a) generating X-ray diffraction data from the crystallized molecule or molecular complex; and

(b) applying at least a portion of the structure coordinates set forth in Table III to the X-ray diffraction pattern to generate a three-dimensional electron density map of at least a portion of the molecule or molecular complex.

Another embodiment of the invention is a method for evaluating the ability of a compound to associate with a molecule or molecular complex comprising a PIN1 PPIase substrate-binding pocket. The method includes the steps of:

(a) constructing a computer model of the binding pocket defined by the structure coordinates of PIN1 PPIase amino acids His59, Leu61, Lys63, Ser67, Arg68, Arg69, Cys113, Leu122, Met130, Gln131, Phe134, Glu135, Thr152, Ser154, and His157 according to Table III;

(b) selecting a compound to be evaluated by a method selected from the group consisting of (i) assembling molecular fragments into a compound, (ii) selecting a compound from a small molecule database, (iii) de novo ligand design of a compound, and (iv) modifying a known modulator, or a portion thereof, of a peptidyl-prolyl isomerase;

(c) employing computational means to perform a fitting program operation between computer models of the compound to be evaluated and the binding pocket in order to provide an energy-minimized configuration of the compound in the binding pocket; and

(d) evaluating the results of the fitting operation to quantify the association between the the compound and the binding pocket model, thereby evaluating the ability of the compound to associate with the binding pocket.

Yet another embodiment of the invention is a method for evaluating the ability of a compound to associate with a molecule or molecular complex comprising a PIN1 PPIase substrate-binding pocket. The method includes the steps of:

(a) constructing a computer model of the binding pocket defined by structure coordinates of PIN1 PPIase amino acids Arg54, Arg56, His59, Leu61, Lys63, Ser67, Arg68, Arg69, Ser72, Trp73, Ser111, Asp112, Cys113, Ser114, Ser115, Ala116, Lys117, Ala118, Arg119, Gly120, Asp121, Leu122, Gly123, Ala124, Phe125, Ser126, Arg127, Gly128, Gln129, Met130, Gln131, Lys132, Pro133, Phe134, Glu135, Thr152, Asp153, Ser154, and His157 according to Table III;

(b) selecting a compound to be evaluated by a method selected from the group consisting of (i) assembling molecular fragments into a compound, (ii) selecting a compound from a small molecule database, (iii) de novo ligand design of a compound, and (iv) modifying a known modulator, or a portion thereof, of a peptidyl-prolyl isomerase;

(c) employing computational means to perform a fitting program operation between computer models of the compound to be evaluated and the binding pocket in order to provide an energy-minimized configuration of the compound in the binding pocket; and

(d) evaluating the results of the fitting operation to quantify the association between the compound and the binding pocket model, thereby evaluating the ability of the compound to associate with the binding pocket.

Also disclosed is a method for identifying a modulator of a molecule comprising a PIN1 PPIase substrate-binding site, including the steps of

(a) constructing a computer model of the the binding pocket defined by structure coordinates of PIN1 PPIase substrate-binding site amino acids His59, Leu61, Lys63, Ser67, Arg68, Arg69, Cys113, Leu122, Met130, Gln131, Phe134, Glu135, Thr152, Ser154, and His157 according to Table III;

(b) selecting a compound to be evaluated as a modulator by a method selected from the group consisting of (i) assembling molecular fragments into a compound, (ii) selecting a compound from a small molecule database, (iii) de novo ligand design of a compound, and (iv) modifying a known inhibitor, or a portion thereof, of a peptidyl-prolyl isomerase;

(c) employing computational means to perform a fitting program operation between computer models of the compound to be evaluated and the binding pocket in order to provide an energy-minimized configuration of the compound in the binding pocket;

(d) evaluating the results of the fitting operation to quantify the association between the compound and the binding pocket model, thereby evaluating the ability of the compound to associate with the binding pocket;

(e) synthesizing the compound; and

(f) contacting the compound with the molecule to determine the ability of the compound to modulate the PPIase activity of the molecule.

A preferred embodiment is a method for identifying a modulator of a molecule comprising a PIN1 PPIase substrate-binding site, including the steps of

(a) constructing a computer model of the binding pocket defined by structure coordinates of PIN1 PPIase amino acids Arg54, Arg56, His59, Leu61, Lys63, Ser67, Arg68, Arg69, Ser72, Trp73, Ser111, Asp112, Cys113, Ser114, Ser115, Ala116, Lys117, Ala118, Arg119, Gly120, Asp121, Leu122, Gly123, Ala124, Phe125, Ser126, Arg127, Gly128, Gln129, Met130, Gln131, Lys132, Pro133, Phe134, Glu135, Thr152, Asp153, Ser154, and His157 according to Table III;

(b) selecting a compound to be evaluated as a potential activator or inhibitor by a method selected from the group consisting of (i) assembling molecular fragments into a compound, (ii) selecting a compound from a small molecule database, (iii) de novo ligand design of a compound, and (iv) modifying a known inhibitor, or a portion thereof, of a peptidyl-prolyl isomerase;

(c) employing computational means to perform a fitting program operation between computer models of the compound to be evaluated and the binding pocket in order to provide an energy-minimized configuration of the compound in the binding pocket;

(d) evaluating the results of the fitting operation to quantify the association between the compound and the binding pocket model, thereby evaluating the ability of the compound to associate with the binding pocket;

(e) synthesizing the compound; and

(f) contacting the compound with the molecule to determine the ability of the compound to modulate the PPIase activity of the molecule.

Another method described herein for screening compounds for PIN1 PPIase modulating activity includes the steps of:

(a) providing an assay buffer containing a Pintide-PIN1 PPIase polypeptide complex;

(b) adding a test compound; and

(c) measuring the disruption of the Pintide-PIN1 PPIase complex.

A preferred embodiment for screening compounds for PIN1 PPIase modulating

activity is a high-throughput screening method that includes the steps of:

(a) providing an assay buffer containing a Pintide-PIN1 PPIase polypeptide complex in a multiple-vessel format, such as a microwell plate;

(b) adding test compounds; and

(c) measuring the disruption of the Pintide-PIN1 PPIase complex in the multiple vessels.

Another preferred embodiment for screening compounds for PIN1 PPIase modulating activity is a high-throughput screening method that includes the steps of:

(a) providing an assay buffer containing a fluorscent-Pintide-PIN1 PPIase polypeptide complex in a multi-vessel format;

(b) adding test compounds; and

(c) measuring the disruption of the fluorscent-Pintide-PIN1 PPIase complex in the multiple vessels.

Yet another preferred embodiment for screening compounds for PIN1 PPIase modulating activity is a high-throughput screening method that includes the steps of:

(a) providing an assay buffer containing a fluorscent-Pintide-PIN1 PPIase polypeptide complex in a multi-vessel format;

(b) adding test compounds; and

(c) measuring the disruption of the fluorscent-Pintide-PIN1 PPIase complex in the multiple vessels using fluorescence-polarization.

### BRIEF DESCRIPTION OF THE DRAWINGS

This patent application file contains at least one drawing executed in color. Copies of this patent application publication with color drawing(s) will be provided by the U.S. Patent and Trademark Office upon request and payment of the necessary fee.

FIG. 1 is a ribbon-and-stick drawing of the PPIase (K77Q/K82Q) domain structure with bound Compound I. Alpha helices are in red, beta strands in yellow, turns in blue, and connecting segments in green. The right-hand panel shows the structure of full-length PIN1.

FIG. 2A shows a close-up view of the PPIase (K77Q/K82Q) active site with Compound I depicted using stick bonds. Amino acid side chains in close proximity to Compound I are represented using stick bonds and colored green.

FIG. 2B shows a close-up view of the PPIase (K77Q/K82Q) active site and the electron density for compound I.

FIG. 3 is a representation of the PPIase (K77Q/K82Q) solvent-accessible surface. Red represents hydrophobic regions and cyan represents hydrophilic regions.

FIG. 4A lists the nucleotide sequence that encodes human PIN1 PPIase domain.

FIG. 4B amino acid sequence of human PIN1 PPIase domain expressed from pET-28a after cleavage with thrombin.

FIG. 5A lists the nucleotide sequence that encodes mutant PPIase K77Q/K82Q.

FIG. 5B lists the amino acid sequence of K77Q/K82Q expressed from pET-28a after cleavage with thrombin.

FIG. 6 is a graphical representation of a calorimetric titration of Compound I with a His-tagged PIN1 PPIase.

### DETAILED DESCRIPTION OF THE INVENTION AND PREFERRED EMBODIMENTS

As used herein, the terms “comprising” and “including” are used in an open, non-limiting sense.

The present invention uses conventional microbiological and recombinant DNA techniques known to those of ordinary skill in the art, See, e.g., Sambrook et al., “Molecular Cloning: A Laboratory Manual,” 3^{rd} ed. (2001) Cold Spring Harbor Press, Cold Spring Harbor, N.Y.; Glover, ed., “DNA Cloning: A Practical Approach,” Volumes I and II, 2^{nd} (1995), IRL Press, Oxford; Ausbel et al., eds. “Current Protocols in Molecular Biology” (1994) Green Publishers Inc. and Wiley and Sons, New York; Innis et al., eds. “PCR Protocols: A Guide to Methods and Applications” (1990) Academic Press, San Diego; Freshney “Culture of Animal Cells: A Manual of Basic Technique,” 4^{th} ed.(2000) Wiley & Sons; and Perbal, “A Practical Guide to Molecular Cloning,” 2^{nd} ed. (1988) Wiley & Sons.

A. Nucleic Acids and Polynucleotides

The present invention provides isolated nucleic acid molecules that encode mutant PIN1 PPIases domains with improved crystallography properties. Such improved properties include the ability to bind ligands better than wild-type PIN1 in a crystallized form, and the ability to be crystallized without phosphate or sulfate. In the absence of phosphate or sulfate, the substrate-binding pocket is more amenable for compound binding.

The terms “nucleic acid molecule” and “polynucleotide” are used interchangeably in this application. These terms refer to any polyribonucleotide or polydeoxribonucleotide, which may be unmodified RNA or DNA or modified RNA or DNA. These terms are intended to include DNA molecules (e.g., cDNA) and RNA molecules (e.g., mRNA) and analogs of the DNA or RNA generated using nucleotide analogs. Exemplary polynucleotides include single- and double-stranded DNA, DNA that is a mixture of single- and double-stranded regions or single-, double- and triple-stranded regions, single- and double-stranded RNA, and RNA that is mixture of single- and double-stranded regions, hybrid molecules comprising DNA and RNA that may be single-stranded or, double-stranded, or triple-stranded regions, or a mixture of single- and double-stranded regions. In addition, “polynucleotide” and “nucleic acid molecule” as used herein refer to triple-stranded regions composed of RNA or DNA, or both RNA and DNA. The strands in such regions may be from the same molecule or from different molecules. The regions may include all of one or more of the molecules, but more preferably involve only a region of some of the molecules. One of the molecules of a triple-helical region may be an oligonucleotide.

Exemplary polynucleotides and nucleic acid molecules also include DNAs or RNAs as described above that contain one or more modified bases. Moreover, DNAs or RNAs comprising unusual bases, such as inosine, or modified bases, such as tritylated bases are exemplary polynucleotides. Exemplary polynucleotides and nucleic acid molecules also include chemically, enzymatically or metabolically modified forms of polynucleotides, as well as the chemical forms of DNA and RNA characteristic of viruses and cells, including, for example, simple and complex cells. Exemplary polynucleotides also include short polynucleotides referred to as oligonucleotides.

As used herein, the term “isolated” nucleic acid molecule means that the material is free of proteins and other nucleic acid present in the natural environment in which the material is normally found. In particular, the nucleic acid molecule is free of cellular components. Exemplary isolated nucleic acid molecules include PCR products, mRNA, cDNA, or restriction fragments. In another embodiment, an isolated nucleic acid is preferably excised from the chromosome in which it may be found, and more preferably is no longer joined to non-regulatory, non-coding regions, or to other genes, located upstream or downstream of the gene in its natural environment in the chromosome. In yet another embodiments the isolated nucleic acid lacks one or more introns. Isolated nucleic acid molecules can be inserted into plasmids, cosmids, artificial chromosomes, and the like. Thus, in a specific embodiment, a recombinant nucleic acid is an isolated nucleic acid. Moreover, an “isolated” nucleic acid molecule, such as a cDNA molecule, can be substantially free of other cellular material, or culture medium when produced by recombinant techniques, or chemical precursors or other chemicals when chemically synthesized. However, the nucleic acid molecule can be fused to other coding or regulatory sequences and still be considered isolated.

For example, a recombinant DNA molecule contained in a vector is considered isolated. Further examples of isolated DNA molecules include recombinant DNA molecules maintained in heterologous host cells or purified (partially or substantially) DNA molecules in solution. Exemplary isolated RNA molecules include in vivo or in vitro RNA transcripts of the isolated DNA molecules described herein. Exemplary isolated nucleic acid molecules further include such molecules produced synthetically.

Full-length genes or portions thereof may be cloned using any one of a number of suitable methods known in the art. For example, a method that employs XL-PCR (Perkin-Elmer, Foster City, Calif.) to amplify long pieces of DNA may be used.

The isolated nucleic acid molecules can encode functional polypeptides plus additional amino or carboxyl-terminal amino acids, such as those that, e.g., facilitate protein trafficking, prolong or shorten protein half-life, or facilitate manipulation of a protein for assay or production. Once a full-length gene is cloned, portions of the gene, such as the PPIase domain, can be obtained using known techniques. The isolated nucleic acid molecules of the invention include the sequence encoding the active PPIase alone or in combination with other coding sequences, such as a leader or secretory sequence (e.g., a pre-pro or pro-protein sequence), the sequence encoding the PPIase domain, with or without the additional coding sequences, plus additional non-coding sequences, for example, introns and non-coding 5′ and 3′ sequences, such as transcribed but non-translated sequences that play a role in transcription, mRNA processing (including splicing and polyadenylation signals), ribosome binding, and stability of mRNA. In addition, the nucleic acid molecule may be fused to a marker sequence encoding, for example, a peptide that facilitates purification.

Isolated nucleic acid molecules can be in the form of RNA, such as mRNA, or in the form of DNA, including cDNA and genomic DNA, obtained by cloning or produced by known chemical synthetic techniques or by a combination thereof. The nucleic acid, especially DNA, can be double-stranded or single-stranded. Single-stranded nucleic acid can be the coding strand (sense strand) or the non-coding strand (antisense strand).

The invention further provides nucleic acid molecules that encode functional fragments or variants of PIN1 PPIases. Such nucleic acid molecules may be constructed by known recombinant DNA methods or by chemical synthesis. Such non-naturally occurring variants may be made by mutagenesis techniques, including those applied to nucleic acid molecules, cells, or organisms. Accordingly, the variants can contain nucleotide substitutions, deletions, inversions and insertions. Variation can occur in either or both the coding and non-coding regions. The variations can produce both conservative and non-conservative amino acid substitutions.

The nucleic acid molecules of the present invention are useful for producing peptides for use in crystallization studies, drug discovery, and drug design. The nucleic acid molecules can also be used as primers for PCR to amplify any given region of a nucleic acid molecule and are also useful to synthesize antisense molecules of desired length and sequence.

The nucleic acid molecules are also useful for constructing recombinant vectors. Such vectors include expression vectors that express a portion of, or all of, the peptide sequences. Vectors also include insertion vectors, used to integrate into another nucleic acid molecule sequence, such as into the cellular genome, to alter in situ expression of a gene and/or gene product. For example, an endogenous coding sequence can be replaced via homologous recombination with all or part of the coding region containing one or more specifically introduced mutations.

The nucleic acid molecules are also useful for constructing host cells expressing a part, or all, of the nucleic acid molecules and peptides.

Vectors and Host Cells

The invention also provides vectors containing the nucleic acid molecules described herein. When the vector is a nucleic acid molecule, the nucleic acid molecules described herein are covalently linked to the vector nucleic acid. Exemplary vectors for this embodiment of the invention include plasmids, single- or double-stranded phage, single- or double-stranded RNA or DNA viral vector, or artificial chromosome, such as a BAC, PAC, YAC, or MAC. Various expression vectors can be used to express the polynucleotides of the invention, such as pET and pProEX.

A vector can be maintained in the host cell as an extrachromosomal element where it replicates and produces additional copies of the nucleic acid molecules. Alternatively, the vector may integrate into the host cell genome and produce additional copies of the nucleic acid molecules when the host cell replicates.

The vectors can be used for the maintenance (cloning vectors) or expression (expression vectors) of the nucleic acid molecules. The vectors can function in prokaryotic or eukaryotic cells or in both (shuttle vectors).

Expression vectors contain cis-acting regulatory regions that are operably linked in the vector to the nucleic acid molecules such that transcription of the nucleic acid molecules is allowed in a host cell. The nucleic acid molecules can be introduced into the host cell with a separate nucleic acid molecule capable of affecting transcription. Thus, the second nucleic acid molecule may provide a trans-acting factor interacting with the cis-regulatory control region to allow transcription of the nucleic acid molecules from the vector. Alternatively, the host cell may supply a trans-acting factor. Finally, a trans-acting factor can be produced from the vector itself. It is understood, however, that in some embodiments, transcription and/or translation of the nucleic acid molecules can occur in a cell-free system.

Exemplary regulatory sequences to which the nucleic acid molecules described herein can be operably linked include promoters for directing mRNA transcription. These include the left promoter from bacteriophage λ, the lac promoter, TRP, and TAC promoters from E. coli, the early and late promoters from SV40, the CMV immediate early promoter, the adenovirus early and late promoters, and retrovirus long-terminal repeats.

The term “operably linked” as used herein indicates that a gene and a regulatory sequence, such as a promoter, are connected in such a way as to permit gene expression when the appropriate molecules (e.g., transcriptional activator proteins or proteins which include transcriptional activation domains) are bound to the regulatory sequence.

In addition to control regions that promote transcription, exemplary expression vectors also include regions that modulate transcription, such as repressor binding sites and enhancers. Illustrative embodiments include the SV40 enhancer, the cytomegalovirus immediate early enhancer, polyoma enhancer, adenovirus enhancers, and retrovirus LTR enhancers.

In addition to containing sites for transcription initiation and control, exemplary expression vectors can contain sequences necessary for transcription termination. These vectors may also contain signals necessary for translation such as a ribosome-binding site. Other exemplary regulatory control elements for expression include initiation and termination codons as well as polyadenylation signals. Other examples of regulatory sequences are described, for example, in Sambrook et al., 2001,supra.

A variety of expression vectors can be used to express a nucleic acid molecule. Examples of such vectors include chromosomal, episomal, and virus-derived vectors, for example, vectors derived from bacterial plasmids, from bacteriophage, from yeast episomes, from yeast chromosomal elements, including yeast artificial chromosomes, and from viruses such as baculoviruses, papovaviruses such as SV40, vaccinia viruses, adenoviruses, poxviruses, pseudorabies viruses, and retroviruses. Vectors may also be derived from combinations of these sources, such as those derived from plasmid and bacteriophage genetic elements, e.g., cosmids and phagemids. Appropriate cloning and expression vectors for prokaryotic and eukaryotic hosts are described in Sambrook et al., 2001, supra.

The regulatory sequence may provide constitutive expression in one or more host cells (i.e. tissue specific) or may provide for inducible expression in one or more cell types such as by temperature, nutrient additive, or exogenous factor such as a hormone or other ligand. Suitable vectors providing for constitutive and inducible expression in prokaryotic and eukaryotic hosts are known in the art.

The nucleic acid molecules can be inserted into the vector nucleic acid by known methodology. For example, the DNA of interest is joined to a vector by cleaving the DNA sequence and the vector with one or more restriction enzymes and then ligating the fragments together.

The vector containing the appropriate nucleic acid molecule can be introduced into an appropriate host cell for propagation or expression using known techniques. Appropriate bacterial host cells include E. coli, Streptomyces, and Salmonella typhimurium. Appropriate eukaryotic host cells include yeast, insect cells, animal cells such as COS and CHO, and plant cells.

In a preferred embodiment, a peptide as described herein is expressed as a fusion protein. Accordingly, the invention also provides fusion vectors that allow for the production of such peptides. Fusion vectors can increase the expression of a recombinant protein, increase the solubility of the recombinant protein, and/or aid in the purification of the protein by acting, for example, as a ligand for affinity purification. A proteolytic cleavage site may be introduced at the junction of the fusion moiety so that the desired peptide can ultimately be separated from the fusion moiety. Exemplary proteolytic enzymes include factor Xa, thrombin, and enterokinase. Illustrative fusion expression vectors include pGEX (Smith et al., Gene 67:31-40 (1988)), pET28a (Novagen, Madison, Wis.), pMAL (New England Biolabs, Beverly, Mass.), and pRIT5 (Pharmacia, Piscataway, N.J.), which fuse glutathione S-transferase (GST), maltose E binding protein, or protein A, respectively, to the target recombinant protein. Examples of suitable inducible non-fusion E. coli expression vectors include pTrc (Amann et al., Gene 69:301-315 (1988)) and pET 11d (Studier et al., Gene Expression Technology: Methods in Enzymology, 185:60-89 (1990)).

Recombinant protein expression can be maximized in a host bacteria by providing a genetic background wherein the host cell has an impaired capacity to proteolytically cleave the recombinant protein. (Gottesman, Gene Expression Technology: Methods in Enzymology, 185:119-128 (1990)). Alternatively, the sequence of the nucleic acid molecule of interest can be altered to provide preferential codon usage for a specific host cell, for example, E. coli. (Wada et al., Nucleic Acids Res. 20:2111-2118 (1992)).

The nucleic acid molecules can also be expressed by expression vectors that are operative in yeast. Examples of vectors for expression in yeast, e.g. S. cerevisiae, include pYepSec1 (Baldari, et al., EMBO J. 6:229-234 (1987)), pMFa (Kurjan et al., Cell 30:933-943 (1982)), pJRY88 (Schultz et al., Gene 54:113-123 (1987)), and pYES2 (Invitrogen Corporation, San Diego, Calif.).

The nucleic acid molecules can also be expressed in insect cells using, for example, baculovirus expression vectors. Exemplary, baculovirus vectors available for expression of proteins in cultured insect cells (e.g., Sf 9 cells) include the pAc series (Smith et al., Mol. Cell Biol. 3:2156-2165 (1983)) and the pVL series (Lucklow et al., Virology 170:31-39 (1989)).

In a preferred embodiment of the invention, the nucleic acid molecules described herein are expressed in mammalian cells using mammalian expression vectors. Examples of mammalian expression vectors include pCDM8 (Seed, Nature 329:840 (1987)) and pMT2PC (Kaufman et al., EMBO J. 6:187-195 (1987)).

Preferred expression vectors include pET28a (Novagen, Madison, Wis.), pAcSG2 (Pharmingen, San Diego, Calif.), pProEx (Life Technologies, Gaithersburg, Md.) and pFastBac (Life Technologies). Other vectors suitable for maintenance propagation or expression of the nucleic acid molecules described herein are known in the art. For example, suitable vectors and methods for using and propagating vectors are discussed in Sambrook et al., 2001, supra.

The invention also relates to recombinant host cells containing the vectors described herein. Exemplary host cells include prokaryotic cells, lower eukaryotic cells such as yeast, other eukaryotic cells such as insect cells, and higher eukaryotic cells such as mammalian cells.

The recombinant host cells are prepared by introducing the vector constructs described herein into the cells by techniques available in the art. These include calcium phosphate transfection, DEAE-dextran-mediated transfection, cationic lipid-mediated transfection, electroporation, transduction, infection, lipofection. See also, Sambrook et al., 2001, supra.

The recombinant host cells expressing the peptides described herein have a variety of uses. For example, the cells are useful for producing the polypeptides of the invention, which can be used for crystallography studies, biochemical studies, and drug discovery.

Host cells can contain more than one vector. Thus, different nucleotide sequences can be introduced on different vectors of the same cell. Similarly, the nucleic acid molecules can be introduced either alone or with other nucleic acid molecules that are not related to the nucleic acid molecules, such as those providing trans-acting factors for expression vectors. When more than one vector is introduced into a cell, the vectors can be introduced independently, co-introduced, or joined to the PPIase polynucleotide vector.

In the case of bacteriophage and viral vectors, these can be introduced into cells as packaged or encapsulated virus by standard procedures for infection and transduction. Viral vectors can be replication-competent or replication-defective. In the case in which viral replication is defective, replication will occur in host cells providing functions that complement the defects.

Exemplary vectors include selectable markers that enable the selection of the subpopulation of cells that contain the recombinant vector constructs. The marker can be contained in the same vector that contains the nucleic acid molecules described herein or may be on a separate vector. Exemplary markers include tetracycline or ampicillin-resistance genes for prokaryotic host cells, and dihydrofolate reductase or neomycin resistance for eukaryotic host cells. However, any marker that provides selection for a phenotypic trait may be used.

B. Peptides, Proteins and Antibodies

The following amino acid abbreviations are used herine: A=Ala=Alanine; V=Val=Valine; L=Leu=Leucine; I=Ile=Isoleucine; P=Pro=Proline; F=Phe=Phenylalanine; W=Trp=Tryptophan; M=Met=Methionine; G=Gly=Glycine; S=Ser=Serine; T=Thr=Threonine; C=Cys=Cysteine; Y=Tyr=Tyrosine; N=Asn=Asparagine; Q=Gln=Glutamine; D=Asp=Aspartic Acid; E=Glu=Glutamic Acid; K=Lys=Lysine; R=Arg=Arginine; and H=His=Histidine.

As used herein, the terms “peptidyl-prolyl isomease” and “PPIase” refer to enzymes that accelerate the cis/trans isomerization of peptide bonds preceding prolyl residues.

The term “mutant PIN1 PPIase” means a polypeptide which contains a PIN1 PPIase domain but which is devoid of the PIN1 WW domain. These mutant PIN1 PPIase polypeptides may also contain discrete amino acid substitutions in their PPIase domain.

“Polypeptide” refers to any peptide or protein comprising two or more amino acids joined to each other by peptide bonds or modified peptide bonds, i.e., peptide isosteres. “Polypeptide” refers to both short chains, commonly referred to as peptides, oligopeptides or oligomers, and to longer chains, generally referred to as proteins. The terms “peptide”, “polypeptide” and “protein” are used interchangeably herein.

As used herein, a peptide is said to be “isolated” or “purified” when it is substantially free of homologous cellular material or chemical precursors or other chemicals. The peptides of the present invention can be purified to homogeneity or other degrees of purity. The level of purification will be selected based on the intended use, such that the preparation allows for the desired function of the peptide, even if in the presence of considerable amounts of other components.

In some embodiments, “substantially free of cellular material” means preparations of the peptide having less than about 30% (by dry weight) other proteins (i.e., contaminating protein). In preferred embodiments the peptide preparation contains less than about 20% other proteins, more preferably less than about 10% other proteins, or even more preferably less than about 5% other proteins. When the peptide is recombinantly produced, it can also be substantially free of culture medium, i.e., culture medium represents less than about 20% of the volume of the protein preparation.

The language “substantially free of chemical precursors or other chemicals” refers to preparations of the peptide in which it is separated from chemical precursors or other chemicals that are involved in its synthesis. The term “substantially free of chemical precursors or other chemicals” means preparations of the mutant PIN1 PPIase polypeptides having less than about 30% (by dry weight) chemical precursors or other chemicals. In preferred embodiments the peptide preparations have less than about 20% chemical precursors or other chemicals, more preferably less than about to 10% chemical precursors or other chemicals, or even more preferably less than about 5% chemical precursors or other chemicals.

The isolated mutant PPIase polypeptides described herein can be purified from cells that have been altered to express it (recombination), or synthesized using known protein synthesis techniques. For example, a nucleic acid molecule encoding the PPIase polypeptide is cloned into an expression vector, the expression vector introduced into a host cell and the protein expressed in the host cell. The protein can then be isolated from the cells by an appropriate purification scheme using standard protein purification techniques.

While the polypeptides of the invention can be produced in bacteria, yeast, mammalian cells, and other cells under the control of the appropriate regulatory sequences, cell-free transcription and translation systems can also be used to produce these proteins using RNA derived from the DNA constructs described herein.

Where secretion of the peptide is desired, appropriate secretion signals are incorporated into the vector. The signal sequence can be endogenous to the peptides or heterologous to these peptides.

It is also understood that, depending upon the host cell in recombinant production of the peptides described herein, the peptides can have various glycosylation patterns, depending upon the cell, or non-glycosylated, as when produced in bacteria. In some embodiments, the peptides may include an initial modified methionine as a result of a host-mediated process.

The present invention also provides variants of the above-described peptides, such as allelic/sequence variants of the peptides, and non-naturally occurring recombinantly derived variants of the peptides. Such variants can be generated using techniques that are known by those skilled in the fields of recombinant nucleic acid technology and protein biochemistry.

Such variants can readily be made or identified using molecular techniques and the sequence information disclosed herein. Further, such variants can readily be distinguished from other peptides based on sequence and/or structural homology to the peptides of the present invention.

To determine the percent identity of two amino acid sequences or two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably 40%, more preferably 50%, even more preferably 60% or more, of the length of the reference sequence. In a preferred embodiment, the length of a reference sequence aligned for comparison purposes is at least 70%, preferably 80%, more preferably 90% or more, of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity and similarity between two sequences can be accomplished using a mathematical algorithm. (Lesk, ed., “Computational Molecular Biology” (1988) Oxford University Press, New York; Smith, ed., “Biocomputing: Informatics and Genome Projects” (1993) Academic Press, New York; Griffin et al., eds., “Computer Analysis of Sequence Data, Part 1” (1994) Humana Press, New Jersey; von Heinje, “Sequence Analysis in Molecular Biology” (1987) Academic Press; and Gribskov et al. eds., “Sequence Analysis Primer” (1991) Stockton Press, New York). For example, the percent identity between two amino acid sequences is determined using the Needleman et al. algorithm (J. Mol. Biol. 48:444-453 (1970), which has been incorporated into commercially available computer programs, such as GAP in the GCG software package, using either a Blossom 62 matrix or a PAM250 matrix, and a gap weight of 16, 14, 12, 10, 8, 6, or 4 and a length weight of 1, 2, 3, 4, 5, or 6. The percent identity between two nucleotide sequences can also be determined using the commercially available computer programs including the GAP program in the GCG software package (Devereux et al., Nucleic Acids Res. 12(1):387 (1984)), the NWS gap DNA CMP matrix and a gap weight of 40, 50, 60, 70, or 80 and a length weight of 1, 2, 3, 4, 5, or 6. The percent identity between two amino acid or nucleotide sequences can be determined using the algorithm of Meyers et al. (CABIOS, 4:11-17 (1989)), which has been incorporated into commercially available computer programs, such as ALIGN (version 2.0), using a PAM120 weight residue table, a gap length penalty of 12 and a gap penalty of 4.

The nucleic acid and protein sequences of the present invention can further be used as a “query sequence” to perform a search against sequence databases to, for example, identify other family members or related sequences. Such searches can be performed using commercially available search engines, such as the NBLAST and XBLAST programs (version 2.0) of Altschul et al. (J. Mol. Biol. 215:403-10 (1990)). Nucleotide searches can be performed with such programs to obtain nucleotide sequences homologous to the nucleic acid molecules of the invention. Protein searches can be performed with such programs to obtain amino acid sequences homologous to the proteins of the invention. To obtain gapped alignments for comparison purposes, Gapped BLAST can be utilized as described in Altschul et al. (Nucleic Acids Res. 25(17):3389-3402 (1997)).

Peptides can be routinely identified as having a high degree (significant) of sequence homology/identity to the peptides of the present invention. As used herein, two proteins (or a region of the proteins) have “significant homology” when the amino acid sequences are typically at least about 70-75% homologous. In preferred embodiments, the homology is 80-85%, and more preferably at least about 90-95%. A significantly homologous amino acid sequence will be encoded by a nucleic acid sequence that will hybridize to a peptide encoding nucleic acid molecule under stringent conditions.

Non-naturally occurring variants of the polypeptides of the present invention can be generated using recombinant techniques. Such variants include deletions, additions and substitutions in the amino acid sequence of the PPIase domain. For example, one class of substitutions are conservative amino acid substitutions. Such substitutions are those that substitute a given amino acid in a peptide by another amino acid of like characteristics. Exemplary conservative substitutions are the replacements, one for another, among the aliphatic amino acids (Ala, Val, Leu, and IIe); interchange of amino acids containing a hydroxyl residue (Ser and Thr); exchange of amino acids containing an acidic residue (Asp and Glu); substitution between amino acids containing an amide residue (Asn and Gln); exchange of amino acids containing a basic residue (Lys and Arg); and replacements among amino acids containing an aromatic residue (Phe, Tyr). Guidance concerning which amino acid changes are likely to be phenotypically silent is found in Bowie et al., Science 247:1306-1310 (1990).

Variant PIN1 PPIases can be fully functional or may have reduced or decreased activity when compared to the wild-type protein. Fully functional variants may contain conservative variation or variation in non-critical residues or in non-critical regions. Functional variants can also contain substitution of similar amino acids, not affecting function that result in no change or an insignificant change in function. Alternatively, such substitutions may positively or negatively affect function to some degree.

Exemplary non-functional variants are those having one or more non-conservative amino acid substitutions, deletions, insertions, inversions, or truncations of the particular polypeptide, or a substitution, insertion, inversion, or deletion in a critical residue or critical region of the polypeptide.

Amino acids that affect function can be identified by methods known in the art, such as site-directed mutagenesis or alanine-scanning mutagenesis (Cunningham et al., 1989, Science 244:1081-1085). The latter procedure introduces single alanine mutations at every residue in the molecule. The resulting mutant molecules are then tested for biological activity, for example, by measuring enzymatic activity. Sites that are critical for binding can also be determined by structural analysis, such as by X-ray crystallography, nuclear magnetic resonance, or photoaffinity labeling (Smith et al., J. Mol. Biol. 224:899-904 (1992); de Vos et al., Science 255:306-312 (1992)). Accordingly, the peptides of the present invention also include derivatives or analogs: in which a substituted amino acid residue is not one encoded by the genetic code; in which a substituent group is included; in which the polypeptide is fused with another compound, such as a compound to increase the half-life of the polypeptide (for example, polyethylene glycol); or in which the additional amino acids are fused to the polypeptide, such as a leader or secretory sequence or a sequence for purification of the polypeptide.

The present invention further provides for functional, active fragments of the PIN1 PPIase domain. A “fragment” is a variant polypeptide having an amino acid sequence that is entirely the same as part but not all of any amino acid sequence of any polypeptide of the invention. As with the mutant PIN1 polypeptides of the invention, fragments may be free-standing or comprised within a larger polypeptide of which they form a part or region; most preferably they are a single continuous region in a single larger polypeptide. As used herein, a “fragment” comprises at least 8 or more contiguous amino acid residues from the protein PPIase domain. Such fragments can be chosen based on the ability to retain the biological activity of the PPIase domain or based on the ability to perform a function, e.g., act as an immunogen. Preferred are fragments that are catalytically active and that have improved crystallography properties as compared to full-length wild-type PIN1. Such fragments will preferably comprise a domain or motif of the PPIase, e.g., active site or binding site.

Polypeptides may contain amino acids other than the 20 amino acids commonly referred to as the 20 naturally occurring amino acids. Further, many amino acids, including the terminal amino acids, may be modified by natural processes, such as byprocessing and other post-translational modifications, or by chemical modification techniques known in the art. Known modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent crosslinks, formation of cystine, formation of pyroglutamate, formylation, gamma carboxylation, glycosylation, GPI anchor formation, hydroxylation, iodination, methylation, myristoylation, oxidation, proteolytic processing, phosphorylation, phenylation, racemization, selenoylation, sulfation, transfer-RNA mediated addition of amino acids to proteins such as arginylation, and ubiquitination. Modifications, such as glycosylation, lipid attachment, sulfation, gamma-carboxylation of glutamic acid residues, hydroxylation and ADP-ribosylation, for instance, are described in most basic texts, such as Creighton, “Proteins-Structure and Molecular Properties,” 2nd ed. (1993) W. H. Freeman and Company, New York. Reviews on this subject include Wold, “Posttranslational Covalent Modification of Proteins,” Johnson, ed., Academic Press, New York 1-12 (1983); Seifter et al. (Meth. Enzymol. 182: 626-646 (1990)); and Rattan et al. (Ann. N.Y. Acad. Sci. 663:48-62 (1992)).

In some embodiments, the peptides can be attached to heterologous sequences to form chimeric or fusion proteins. Such chimeric and fusion proteins comprise a peptide operatively linked to a heterologous protein having an amino acid sequence not substantially homologous to the PPIase peptide. “Operatively linked” indicates that the peptide and the heterologous protein are fused in-frame. The heterologous protein can be fused to the N-terminus or C-terminus of the PPIase peptide. The two peptides linked in a fusion peptide are preferrably derived from two independent sources, and therefore such a fusion peptide comprises two linked peptides not normally found linked in nature.

In some embodiments, the fusion protein does not affect the activity of the peptide per se. For example, the fusion protein can include, enzymatic fusion proteins or affinity tags, for example, beta-galactosidase fusions, yeast two-hybrid GAL fusions, His-tags, MYC-tags, green fusion protein, and Ig fusions. Such fusion proteins can facilitate the purification of the polypeptides described herein. In certain host cells (e.g., mammalian host cells), expression and/or secretion of a protein can be increased by using a heterologous signal sequence.

A chimeric or fusion protein can be produced by standard recombinant DNA techniques. For example, DNA fragments coding for the different protein sequences are ligated together in-frame in accordance with conventional techniques. In another embodiment, the fusion gene can be synthesized by conventional techniques, including automated DNA synthesizers. Alternatively, PCR amplification of gene fragments can be carried out using anchor primers which give rise to complementary overhangs between two consecutive gene fragments, which can subsequently be annealed and re-amplified to generate a chimeric gene sequence (see Ausubel et al., 1992 supra). Moreover, many expression vectors are commercially available that already encode a fusion moiety (e.g., a GST protein, His-tag, or green fluorescent protein). A nucleic acid encoding a PPIase polypeptide can be cloned into such an expression vector such that the fusion moiety is linked in-frame to the PPIase polypeptide.

The polypeptides can be used for rapid-screening methods (high-throughput screening) to identify compounds that inhibit or modulate PIN1 PPIase activity. The high-throughput screening assay can be fully automated on robotic workstations. The assay may employ radioactivity, fluorescence, or other materials useful for detection.

“High-throughput screening” as used herein refers to an assay that provides for multiple-candidate agents or samples to be screened simultaneously. Preferably the number of agents or samples screened is greater than one, more preferably greater than 100, and even more preferably greater than 300. Such assays may include the use of microtiter plates or other vessel containing apparatus that allows a large number of assays to be carried out simultaneously, using small amounts of reagents and samples.

C. Crystallization and Drug Design

Crystals of the polypeptides of the invention or ligand complexes of such polypeptides can be grown by a number of known techniques, including batch crystallization, vapor diffusion (either by sitting drop or hanging drop), and microdialysis. Seeding of the crystals in some instances is required to obtain X-ray quality crystals. Standard micro and/or macro seeding of crystals may therefore be used. As exemplified below, PIN1 PPIase-Compound I complex was prepared by diluting PIN1 PPIase to 10 mg/ml, then exposing it to Compound I dissolved in 100% DMSO to a final concentration of 1 mM. The resulting protein/Compound I solution was then incubated for 24 hours at 4° C., and filtered through a 0.45-μM cellulose-acetate membrane prior to setting up crystallization experiments. Under these conditions, crystals grew within 3 days.

Once a crystal of the present invention is grown, X-ray diffraction data can be collected. X-ray diffraction data collection can be obtained using, for example, an MAR-imaging plate detector. Crystals can be characterized by using X-rays produced in a conventional source (such as a sealed tube or a rotating anode) or using a synchrotron source (provided by, e.g., the Stanford University Synchrotron Radiation Laboratory).

Data processing and reduction can be carried out using programs such as DENZO/SCALEPACK (HKL Research, Inc., Charlottesvilee, Va.; Otwinowski et al., Meth. Enzymol. 276:307-326 (1997)). In addition, X-PLOR (Brunger, “X-PLOR:A System for X-ray Crystallography and NMR,” Yale University Press, New Haven, Conn (1992)) or Heavy (Terwilliger, Los Alamos National Laboratory) may be utilized for bulk solvent correction and B-factor scaling. Electron density maps can be calculated using SHARP (La Fortelle et al., Meth. Enzymol. 276:472-494 (1997)) and SOLOMON (Abrahams et al., Acta Cryst. D52:30-42 (1996)). Molecular models can be built into this map using 0 (Jones et al., ACTA Crystallogr. A47:110-119 (1991)), XTALVIEW (Scripps Research, La Jolla, Calif.) or QUANTA98 (Accelrys, Inc. San Diego, Calif.). Refinement can be done using X-PLOR (Brunger, 1992, supra,), using the free R-value to monitor the course of refinement.

Once the three-dimensional structure of a crystal comprising a PIN1 PPIase or a PIN1 PPIase-complex is determined, a potential ligand (antagonist or agonist) is examined through the use of computer modeling using a docking program such as FelxiDock (Tripos, St. Louis, Mo.), GRAM (Medical Univ. Of South Carolina), DOCK (Univ. of California at San Francisco), Glide (Schrödinger, Portland, Oreg.), Gold (Cambridge Crystallographic Data Centre, UK), FlexX (BioSolveIT GmbH, Germany); AGDOCK (Gehlhaar et al., Chemistry & Biol. 2:317-324 (1995); Bouzida et al., Pacific Symp. on Biocomputing '99, 426-437 (1999); Bouzida et al., Internat. J of Quantum Chem. 72:73-84 (1999); Gehlhaar et al., Proceedings of the Seventh Ann. Conf on Evolutionary Programming, The MIT Press, Cambridge, Mass. (1998); Hex (Ritchie et al., Proteins: Struct. Funct. & Genet. 39:178-194 (2000); all incorporated herein by refernce), or AUTODOCK (Scripps Research Institute, La Jolla, Calif.). This modeling procedure can include computer fitting of potential ligands to the PPIase substrate-binding domain to ascertain how well the shape and the chemical structure of the potential ligand will complement or interfere with the PPIase substrate-binding domain (Bugg et al., Scientific American Dec.:92-98 (1993); West et al., TIPS, 16:67-74 (1995)).

Computer programs can also be employed to estimate the attraction, repulsion, and steric hindrance of the ligand to the PPIase-binding domain. For example, one can screen computationally small molecule databases for chemical entities or compounds that can bind in whole, or in part, to PIN1 PPIase. In this screening, the quality of fit of such entities or compounds to the binding site may be judged either by shape complementarity or by estimated interaction energy (Meng, et al., J. Comp. Chem., 13:505-524 (1992)). Generally, the tighter the fit (e.g., the lower the steric hindrance and/or the greater the attractive force), the more potent the drug is projected to be since these properties are consistent with a tighter-binding constant.

“Binding domain,” also referred to as “binding site,” “binding pocket,” “substrate-binding site,” “catalytic domain,” or “substrate-binding domain,” refers to a region or regions of a molecule or molecular complex, that, as a result of its shape, can associate with another chemical entity or compound. Such regions are of utility in fields such as drug discovery. The association of natural ligands or substrates with binding pockets of their corresponding receptors or enzymes is the basis of many biological mechanisms of action. Similarly, many drugs exert their biological effects via an interaction with the binding pockets of a receptor or enzyme. Such interactions may occur with all or part of the binding pocket. An understanding of such interactions can facilitate the design of drugs having more favorable and specific interactions with their target receptor or enzyme and, thus, improved biological effects. Therefore, information related to ligand binding with the PIN1 substrate-binding site is valuable in facilitating the design and discovery of modulators of PIN1. Furthermore, the more specificity in the design of a potential drug the more likely that the drug will not interact with other similar proteins, thus minimizing potential side effects due to unwanted cross interactions.

Initially, a potential ligand can be obtained by screening a random chemical library. A ligand selected in this manner could be then be systematically modified by computer-modeling programs until one or more promising potential ligands are identified. Such analysis has been shown to be useful in the design of, for example, HIV protease inhibitors (Lam et al., Science 263:380-384 (1994); Wlodawer et al., Ann. Rev. Biochem. 62:543-585 (1993); Appelt, Perspectives in Drug Discovery and Design 1:23-48 (1993); Erickson, Perspectives in Drug Discovery and Design 1: 109-128 (1993). Additionally, directed or focused libraries can be constructed as a means of modifying compounds previously identified as ligands from screening a random chemical library. Using this method, a number of different compounds can be synthesized that systematically explore a particular portion of the ligand-binding site and then tested for activity against the protein of interest. For example, in compound I, the phenyl group could be replaced with substituents that have different physical and chemical properties than the phenyl group.

Such computer modeling allows the selection of a finite number of rational chemical modifications, as opposed to the potentially unlimited number of essentially random chemical modifications that could be made, any one of which might lead to a drug. Each chemical modification requires additional chemical steps, which, while being reasonable for the synthesis of a finite number of compounds, quickly becomes overwhelming if all possible modifications needed to be synthesized. Thus, through the use of the structure coordinates disclosed herein and computer modeling, a large number of these compounds can be rapidly modeled via a computer, and a few promising candidates can be determined without the laborious synthesis of a multitude of compounds.

Once a potential ligand (agonist or antagonist) is identified, it can be either selected from commercial libraries of compounds or alternatively the potential ligand may be synthesized de novo. The prospective drug can be tested in the binding assay exemplified below to test its ability to bind to the PPIase substrate-binding domain, or it can be tested for its ability to modulate PIN1 PPIase activity.

The term “modulates” refers to the ability of a compound to alter the function of a peptidyl-prolyl isomerase, such as PIN1. For example, a compound modulates the activity of a peptidyl-prolyl isomerase if it either increases or decreases the peptidyl-prolyl isomerase activity of the peptidyl-prolyl isomerase protein.

When a suitable compound is identified, a supplemental crystal can be grown that comprises a protein-ligand complex formed between the PIN1 PPIase domain and the compound. Preferably, the crystal effectively diffracts X-rays allowing the determination of the atomic coordinates of the protein-ligand complex to a resolution of greater than or equal to 3.0 Å, more preferably greater than or equal to 2.0 Å. Molecular Replacement Analysis can be used to determine the three-dimensional structure of the supplemental crystal.

Molecular replacement involves using a known three-dimensional structure as a search model to determine the structure of an identical or closely related molecule or protein-ligand complex in a new crystal form. The measured X-ray diffraction properties of the new crystal are compared with those calculated from the search model structure to compute the position and orientation of the protein in the new crystal. Computer programs that can be used for this purpose include: X-PLOR (Brunger, 1992, supra, EPMR (Kissinger et al. Acta Cryst. D55:484-491 (1999); incorporated herein by refernce), ProLSQ (Konnert et al., Acta Cryst. A36:344-350 (1980)), and AMORE (J. Navaza, Acta Crystallographics ASO, 157-163 (1994)). Once the position and orientation are known, an electron density map can be calculated using the search model to provide X-ray phases. Thereafter, the electron density is inspected for structural differences and the search model is modified to conform to the new structure. Using this approach, the structure may be used to solve the three-dimensional structures of any such PIN1 PPIase polypeptide-ligand complex. Other computer programs that can be used to solve the structures of such PIN1 PPIase crystals include QUANTA (Accelrys, Inc., San Diego, Calif.), INSIGHT (Accelrys, Inc., San Diego, Calif.), ARP/wARP (European Molecular Biology Laboratory, Heidelberg, Germany; Perrakis et al., Nature Struc. Biol. 6:458-463 (1999); Lamzin et al., Acta Cryst.D49:129-147 (1993)), and ICM (MolSoft, La Jolla, Calif.)

For all of the drug design strategies described herein, successive iterations of any and/or all of the steps provided by the aforementioned procedures are typically performed to yield one or more ligands with improved properties (e.g., activity).

Another aspect of the invention involves using the structure coordinates generated from the PPIase-ligand complex to generate a three-dimensional shape. This is achieved through the use of commercially available software that is capable of generating three-dimensional graphical representations of molecules or portions thereof from a set of structure coordinates.

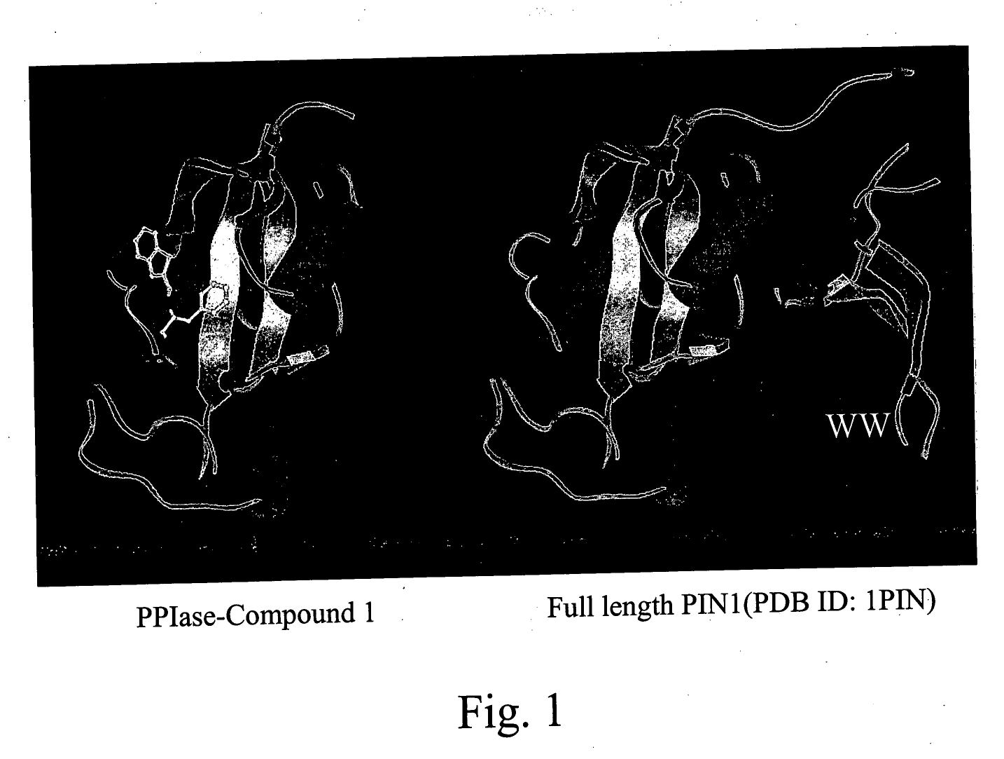

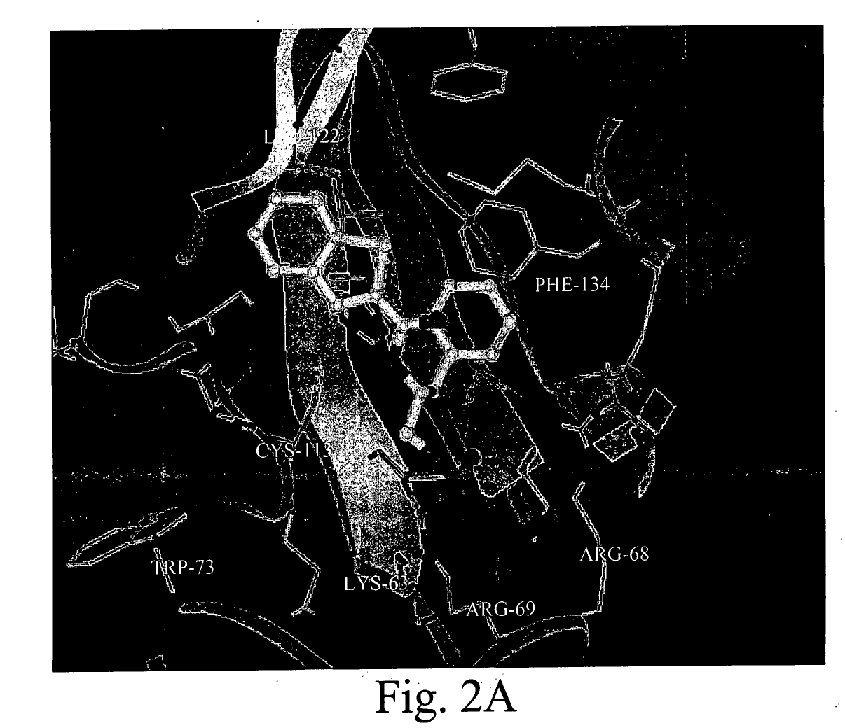

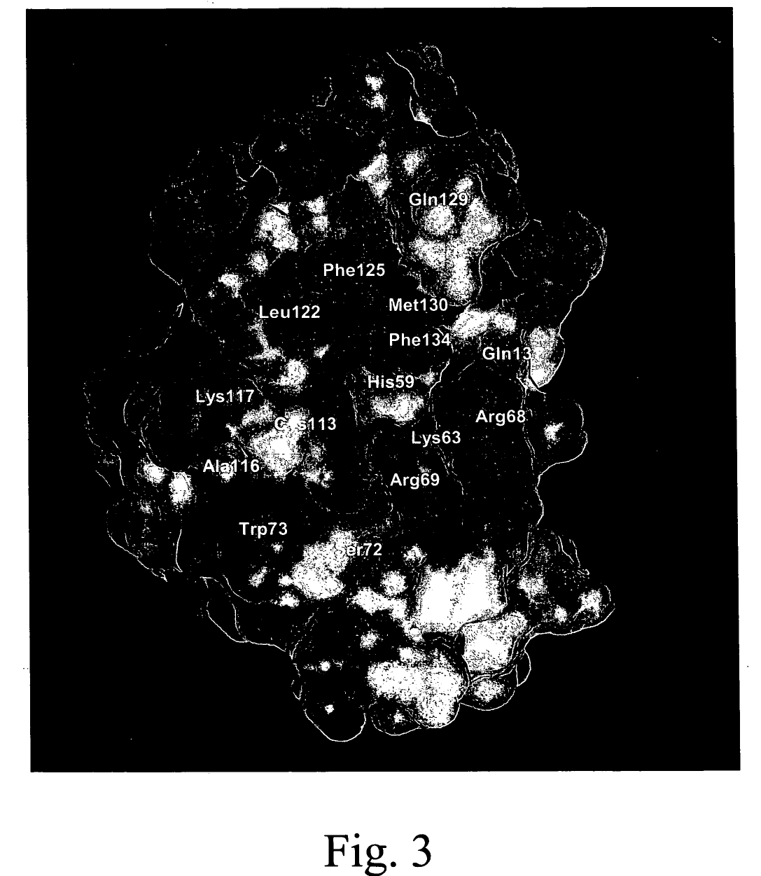

In resolving the crystal structure of a mutant PIN1 PPIase polypeptide as described below, the PIN1 amino acids that define the shape of the PIN1 PPIase substrate-binding domain were determined. For example, one component of the PPIase substrate-binding domain is the surface formed by amino acids Leu61, Cys113, Ser114, Ser115, Ala116, Lys117, Ala118, Arg119, Gly120, Asp121, Leu122, Gly123, Ala124, Phe125, Ser126, Arg127, Gly128, Gln129, and Met130. These residues play a part in binding (hydrophobic interaction). Arg54, Lys117, and Gln129 can also form electrostatic interactions with entities that bind in the PIN1 PPIase substrate-binding site. Arg54, Arg56, Ser111, Lys132, and Asp153, although slightly away from the direct ligand interaction, could interact with modified or larger ligands. Additionally, the prolyl pocket includes His59, Leu122, Phe134, Met130, His157, Thr152, Ser154, Gln131, and Cys113. Lys63, Ser67, Arg68 and Arg69 are relevant to electrostatic interactions. The interaction of Lys63 and Ser67 can be direct or indirect, such as with water mediation. Further, the crystal structure indicates a Gln131 pocket, with potential interaction to Gln131, Thr152, Glu135, and Pro133. Still further, a Trp73 pocket is formed by amino acids Arg69; Ser114, Ser72, Trp73, Asp112 and Ala116. There is a potential covalent adduct to Cys113. Thus, a binding pocket defined by the structural coordinates of these amino acids, as set forth in Table III, or a binding pocket whose root-mean-square deviation from the structure coordinates of the backbone atoms of these amino acids that is not more than about 0.5 Å, is a PIN1 PPIase or PPIase-like substrate-binding domain of this invention. Depictions of the PIN1 PPIase substrate-binding site are shown in FIGS. 1-3.

It will be readily apparent to those of skill in the art that the numbering of amino acids in other isoforms of PIN1 may be different than that set forth herein. Corresponding amino acids in other isoforms of PIN1 are readily identified by inspection of the amino acid sequences, for example, through the use of commercially available homology software programs.

The amino acids of the PPIase domain of the polypeptides of the invention are described herein in reference to the set of structure coordinates set forth in Tables II and III. The terms “structure coordinates” and “atomic coordinates” refer to Cartesian coordinates derived from mathematical equations related to the patterns obtained on diffraction of a monochromatic beam of X-rays by the atoms (scattering centers) of a protein or protein-ligand complex in crystal form. The diffraction data are used to calculate an electron density map of the repeating unit of the crystal. The electron density maps are then used to establish the positions of the individual atoms of the enzyme or enzyme complex.

The variations in coordinates discussed above may be generated because of mathematical manipulations of the PIN1 PPIase-Compound I complex structure coordinates. For example, the structure coordinates set forth in Table III may be manipulated by crystallographic permutations of the structure coordinates, fractionalization of the structure coordinates, integer additions, subtractions to sets of the structure coordinates, coordinate transformations, e.g., translation or rotation, or combinations thereof.

Alternatively, modifications in the crystal structure due to mutations, additions, substitutions, and/or deletions of amino acids, or other changes in any of the components that make up the crystal may also account for variations in structure coordinates. If such variations are within an acceptable standard error as compared to the original coordinates, the resulting three-dimensional shape is considered to be the same. Thus, for example, a ligand that has bound to the binding pocket of the mutant PPIase domain would also be expected to bind to another binding pocket whose structure coordinates, when compared to those described, have a root-mean-square difference of equal to or less than about 0.5 Å from the backbone atoms.

Various computational analyses can be performed to determine whether a polypeptide or the binding pocket portion thereof is sufficiently similar to the PPIase binding pocket as described herein. Such analyses may be carried out through the use of known software applications, such as the MODELLER module of INSIGHT II (Accelrys, Inc., San Diego, Calif.), ProMod (University of Geneva, Switzerland), SWISS-MODEL (Swiss Institute of Bioinformatics), and the Molecular Similarity application of QUANTA (Accelrys, Inc., San Diego, Calif.).

Programs such as QUANTA (Accelrys, Inc., San Diego, Calif.), INSIGHT II (Acceirys, Inc., San Diego, Calif.), Maestro (Schrödinger, Portland, Oreg.), SYBYL (Tripos, Inc., St. Louis, Mo.), and MacroModel (Schrodinger, Portland, Oreg.) permit comparisons between different structures, different conformations of the same structure, and different parts of the same structure. Comparison of structures using such computer software may involve the following steps: 1) loading the structures to be compared; 2) defining the atom equivalencies in the structures; 3) performing a fitting operation; and 4) analyzing the results.

In comparing structures, each structure is identified by a name. One structure is identified as the target (i.e., the fixed structure); all remaining structures are working structures (i.e., moving structures). Since atom equivalency with QUANTA is defined by user input, as defined herein “equivalent atoms” refers to protein backbone atoms (N, Cα, C, and O) for all conserved residues between the two structures being compared.

When a rigid-fitting method is used, the working structure is translated and rotated to obtain an optimum fit with the target structure. The fitting operation uses an algorithm that computes the optimum translation and rotation to be applied to the moving structure, such that the root-mean-square difference of the fit over the specified pairs of equivalent atoms is an absolute minimum. This number, given in angstroms (Å), is reported by software applications such as QUANTA (Accelrys, Inc., San Diego, Calif.) or other similar programs. Any molecule or molecular complex or binding pocket thereof that has a root-mean-square deviation of conserved residue backbone atoms (N, Cα, C, O) of less than about 0.5 Å when superimposed on the relevant backbone atoms described by structure coordinates listed in Table III are considered identical.

The term “root-mean-square deviation” means the square root of the arithmetic mean of the squares of the deviations from the mean. It is a way to express the deviation or variation from a trend or object. As used herein, the “root-mean-square deviation” defines the variation in the backbone of a protein from the backbone of the PIN1 PPIase polypeptides of the invention or the PIN1 PPIase substrate-binding domain portion thereof, as defined by the structure coordinates described herein.

D. Computers, Computer Software, Computer Modeling

As discussed above, a computer may be used for producing a three-dimensional representation of the PPIase substrate-binding domain. Suitable computers are known in the art and typically include a central processing unit (CPU), and a working memory, which can be random-access memory, core memory, mass-storage memory, or a combination thereof. The CPU may encode one or more programs. Computers also typically include display, input and output devices, such as one or more cathode-ray tube display terminals, keyboards, modems, input lines and output lines. Further, computers may be networked to computer servers (the machine on which large calculations can be run in batch) and file servers (the main machine for all the centralized databases).

Machine-readable media containing data, such as the crystal structure coordinates of the polypeptides, may be inputted using various hardware, including modems, CD-ROM drives, disk drives, or keyboards.

Machine-readable data medium can be, for example, a floppy diskette, hard disk, or an optically-readable readable data storage medium, which can be either read only memory, or rewritable, such as a magneto-optical disk.

Output hardware, such as a CRT display terminal, may be used for displaying a graphical representation of the substrate-binding site of the PPIase polypeptides described herein. Output hardware may also include a printer and disk drives.

The CPU coordinates the use of the various input and output devices, coordinates data accesses from storage and accesses to and from working memory, and determines the sequence of data processing steps. A number of programs may be used to process the machine-readable data. Such programs are discussed herein in reference to the computational methods of drug discovery.

In a preferred embodiment of the invention, X-ray coordinate data capable of being processed into a three-dimensional graphical display of a molecule or molecular complex that comprises a PPIase or PPIase-like substrate-binding pocket are stored in a machine-readable storage medium. The three-dimensional structure of a molecule or molecular complex comprising a PPIase or PPIase-like substrate-binding pocket is useful for a variety of purposes in drug discovery and drug design.

For example, the three-dimensional structure derived from the structure coordinate data may be computationally evaluated (computer-aided drug design) for its ability to associate with chemical entities (Butt et al., Scientific American Dec.:92-98 (1993); West et al., TIPS 16:67-74 (1995); Dunbrack et al., Folding & DesignI 2:27-42 (1997)). The term “chemical entity,” as used herein, refers to a chemical compound, a complex of at least two chemical compounds, or a fragment of such a compound or complex. Such entities are potential drug candidates and can be evaluated for their ability to inhibit or modulate the activity of PIN1. The ability of an entity to bind to, or associate with a PIN1 PPIase or PPIase-like substrate-binding domain, depends on the features of the entity alone. Assays to determine if a compound binds to PIN1 are known in the art, such as those exemplified herein.

The design of compounds that bind to a PIN1 PPIase or PPIase-like substrate-binding domain may involve consideration of two factors. First, the entity must be capable of physically and structurally associating with some or the entire PIN1 PPIase or PPIase-like substrate-binding domain. The term “associating with” refers to a condition of proximity between a chemical entity and a binding pocket or binding site on a protein. The association may be non-covalent, for example, wherein the juxtaposition is energetically favored by hydrogen bonding of van der Waals or electrostatic interactions, or it may be covalent. Non-covalent molecular interactions contributing to this association include hydrogen bonding, van der Waals interactions, hydrophobic interactions, and electrostatic interactions.

Second, the entity must be able to assume a conformation that allows it to associate with the PIN1 PPIase or PPIase-like substrate-binding domain directly. Although certain portions of the entity will not directly participate in these associations, those portions of the entity may still influence the overall conformation of the molecule. This, in turn, may have a significant impact on potency. Such conformational requirements include the overall three-dimensional structure and orientation of the chemical entity in relation to all or a portion of the binding pocket, and the spacing between functional groups of an entity comprising several chemical entities that directly interact with the PIN1 PPIase or PPIase-like binding pocket.

The potential inhibitory or binding effect of a chemical entity on a PIN1 PPIase or PPIase-like substrate-binding domain may be analyzed prior to its actual synthesis and testing through the use of computer-modeling techniques. If from the theoretical structure of the given entity it can be surmised that there is insufficient interaction and association between it and the PIN1 PPIase or PPIase-like-binding pocket, further testing of the entity may not be prudent. However, if computer modeling indicates a strong interaction, the molecule can be synthesized and tested for its ability to bind to a PIN1 PPIase or PPIase-like binding pocket. This may be achieved by testing the ability of the molecule to modulate PIN1 PPIase activity using the assays described in herein. Using this scheme, the fruitless synthesis of compounds with poor binding activities may be avoided.

A potential inhibitor of a PIN1 PPIase or PPIase-like substrate-binding domain may be computationally evaluated (computer-aided drug design) by means of a series of steps in which chemical entities are screened and selected for their ability to associate with the PIN1 PPIase or PPIase-like binding pockets. One skilled in the art may use one of several methods to screen chemical entities or fragments for their ability to associate with a PIN1 PPIase or PPIase-like substrate-binding domain. For example, the artesian may visually inspect a PIN1 PPIase or PPIase-like substrate-binding pocket on a computer screen based on the PIN1 PPIase structure coordinates reported in Table III or other coordinates that define a similar shape generated from the machine-readable storage medium. Selected chemical entities may then be positioned in a variety of orientations, or docked, within that binding pocket as described herein. Docking may be accomplished using software such as Quanta (Accelrys, Inc., San Diego, Calif.) and SYBYL (Tripos, Inc., St. Louis, Mo.), followed by energy minimization and molecular dynamics with standard molecular mechanics force fields, such as CHARMM (Department of Chemistry & Chemical Biology, Harvard Univ., Cambridge, Mass.) and AMBER (School of Pharmacy, Department of Pharmaceutical Chemistry, University of California at San Francisco, Calif.)

Specialized computer programs to assist in the process of selecting chemical entities include those described in the following references, which are incorporated by reference herein:

1. GRID (Goodford, “A Computational Procedure for Determining Energetically Favorable Binding Sites on Biologically Important Macromolecules,” J. Med. Chem. 28:849-857 (1985)). GRID is available from the Oxford University, Oxford, UK.

2. MCSS (Miranker et al., “Functionality Maps of Binding Sites: A Multiple Copy Simultaneous Search Method,” Proteins: Struct. Funct. and Genet. 11:29-34 (1991)). MCSS is available from Accelrys, Inc., San Diego, Calif.

3. AUTODOCK (Goodsell et al., “Automated Docking of Substrates to Proteins by Simulated Annealing”, Proteins: Struct. Funct. and Genet. 8:195-20 (1990)). AUTODOCK is available from the Scripps Research Institute, La Jolla, Calif.

4. DOCK (Kuntz et al., “A Geometric Approach to Macromolecule-Ligand Interactions,” J. Mol. Biol., 161:269-288 (1982)). DOCK is available from the University of California, San Francisco, Calif.

5. GOLD (Jones et al., “Development and Validation of a Genetic Algorithm for Flexible Docking,” J. Mol. Biol 267:727-748 (1997)). GOLD is available from the Cambridge Crystallographic Data Centre, UK.

6. GLIDE (Eldridge et al., “Empirical Scoring Functions: I. The Development of a Fast Empirical Scoring Function to Estimate the Binding Affinity of Ligands in Receptor Complexes,” J. Comput. Aided Mol. Des. 11:425-445 (1997)). Glide is available from Schrödinger, Portland Oreg.

Once suitable chemical entities have been selected, they can be assembled into a single compound or complex. Assembly may be preceded by visual inspection of the relationship of the fragments to each other on the three-dimensional image displayed on a computer screen in relation to the structure coordinates of PIN1 PPIase or a PIN1 PPIase-ligand complex. This can be followed by manual model building using software such as Quanta or SYBYL. Useful programs to aid one of skill in the art in connecting the individual chemical entities also include those described in the following references, which are incorporated by reference herein:

1. CAVEAT (Bartlett et al., “CAVEAT: A Program to Facilitate the Structure-Derived Design of Biologically Active Molecules”, Molecular Recognition in Chemical and Biological Problems”, Special Pub., Royal Chem. Soc., 78, pp. 182-196 (1989); Lauri et al., “CAVEAT: a Program to Facilitate the Design of Organic Molecules”, J. Comput. Aided Mol. Des. 8:51-66 (1994)). CAVEAT is available from the University of California, Berkeley, Calif.

2. ISIS: See Martin, “3D Database Searching in Drug Design,” J. Med. Chem. 35:2145-2154 (1992)). ISIS is available from MDL Information Systems, San Leandro, Calif.

3. HOOK (Eisen et al., “HOOK: A Program for Finding Novel Molecular Architectures that Satisfy the Chemical and Steric Requirements of a Macromolecule Binding Site,” Proteins: Struct., Funct., Genet., 19:199-221 (1994)). HOOK is available from Accelrys, Inc., San Diego, Calif.

Instead of proceeding to build an inhibitor of a PIN1 PPIase or PPIase-like substrate-binding pocket in a step-wise fashion one chemical entity at a time as described above, inhibitory or other PIN1 PPIase-binding compounds may be designed as a whole or de novo using either an empty binding site or optionally including some portion(s) of a known inhibitor(s). There are many known de novo ligand design methods, such as LeapFrog (available from Tripos Associates, St. Louis, Mo.) and those discussed in the following references, which are incorporated by reference herein.

1. LUDI (Bohm, “The Computer Program LUDI: A New Method for the De novo Design of Enzyme Inhibitors,” J. Comp. Aid. Molec. Design. 6:61-78 (1992)). LUDI is available from Accelrys Inc., San Diego, Calif.

2. SPROUT (Gillet et al., “SPROUT: A Program for Structure Generation,” J. Comput. Aided Mol. Design. 7:127-153 (1993)). SPROUT is available from the University of Leeds, UK.

Other molecular modeling techniques may also be employed (see, e.g., Cohen et al., J. Med Chem. 33:883-894 (1990); Navia et al., Curr. Opin. Struct. Biol. 2:202-210 (1992); Balbes et al., Reviews in Computational Chemistry, Vol. 5, K. Lipkowitz et al., eds., VCH, New York, pp. 337-380 (1994); Guida, Curr. Opin. Struct. Biol. 4:777-781 (1994)).

Once a chemical entity has been designed or selected by using such methods, the efficiency with which that entity may bind to a PIN1 PPIase substrate-binding pocket may be tested and optimized by computational evaluation. For example, an effective PIN1 PPIase substrate-binding-pocket inhibitor preferably demonstrates a relatively small difference in energy between its bound and free states (i.e., a small deformation energy of binding). PIN1 PPIase substrate-binding pocket inhibitors may interact with the substrate-binding domain in more than one conformation that is similar in overall binding energy. In those cases, the deformation energy of binding is taken to be the difference between the energy of the free entity and the average energy of the conformations observed when the inhibitor binds to the protein.

An entity designed or selected as binding to a PIN1 PPIase substrate-binding domain may be further computationally optimized so that in its bound state it would preferably lack repulsive electrostatic interaction with the target enzyme and with the surrounding water molecules. Such non-complementary electrostatic interactions include repulsive charge-charge, dipole-dipole and charge-dipole interactions.

Suitable computer software is available to evaluate compound deformation energy and electrostatic interactions. Examples of programs designed for such uses include: Gaussian (Frisch, Gaussian, Inc., Carnegie, Pa.); AMBER (Kollman, University of California at San Francisco); Jaguar (Schrödinger, Portland, Oreg.); SPARTAN (Wavefunction, Inc., Irvine, Calif.); QUANTA/CHARMM (Accelrys, Inc., San Diego, Calif.); Impact (Schrödinger, Portland, Oreg.); Insight II/Discover (Accelrys, Inc., San Diego, Calif.); MacroModel (Schrödinger, Portland, Oreg.); Maestro (Schrödinger, Portland, Oreg.); DelPhi (Accelrys, Inc., San Diego, Calif.); and AMSOL (Quantum Chemistry Program Exchange, Indiana University). These programs may be implemented, for instance, using workstations produced by companies, such as Silicone Graphics, Hewlet Packard, Sun Microsystems, and International Business Machines.

In another approach small-molecule databases are computationally screened to determine their potential to bind in whole, or in part, to a PIN1 PPIase or PPIase-like substrate-binding pocket. In this screening, the quality of fit of such entities to the binding site may be judged either by shape complementarity or by estimated interaction energy (Meng et al. J. Comp. Chem. 13:505-524 (1992)). Binding of potential modulators can be assessed biochemically, for example, using isothermal titration calorimetry as described herein.

The structure coordinates set forth in Table III can be used to obtain structural information about another crystallized molecule or molecular complex. This may be achieved by any suitable known technique, such as molecular replacement. By using molecular replacement, all or part of the structure coordinates of the mutant PIN1 PPIase polypeptide:Compound I complex can be used to determine the structure of a crystallized molecule or molecular complex whose structure is unknown. This process is more efficient than attempting to determine such information ab initio.

Molecular replacement provides an accurate estimation of the phases for an unknown structure. Phases constitute a factor in equations used to solve crystal structures that cannot be determined directly. Obtaining accurate values for the phases, by methods other than molecular replacement, is a time-consuming process that involves iterative cycles of approximations and refinements and greatly hinders the solution of crystal structures. However, when the crystal structure of a protein containing at least a homologous portion has been solved, the phases from the known structure can provide a an estimate of the phases for the unknown structure.

The method involves generating a preliminary model of a molecule or molecular complex whose structure coordinates are unknown, by orienting and positioning the relevant portion of the mutant PIN1 PPIase:Compound I complex according to Table III within the unit cell of the crystal of the unknown molecule or molecular complex so as best to theoretically account for the observed X-ray diffraction data of the crystal of the molecule or molecular complex whose structure is unknown. Phases can then be calculated from this model and combined with the observed X-ray diffraction data amplitudes to generate an electron density map of the structure whose coordinates are unknown. This, in turn, can be subjected to any known model building and structure refinement techniques to provide a final, accurate structure of the unknown crystallized molecule or molecular complex (Lattman, Meth. Enzymol. 115:55-77 (1985); Rossmann, ed., “The Molecular Replacement Method,” Int. Sci. Rev. Ser., No. 13, Gordon & Breach, New York (1972)). Thus, the structure of any portion of any crystallized molecule or molecular complex that is sufficiently homologous to any portion of the mutant PIN1 PPIase:Compound I complex can be resolved by this method.

In another preferred embodiment, the method of molecular replacement is utilized to obtain structural information about another PPIase. The structure coordinates of PIN1 PPIase as described herein are useful in solving the structure of other isoforms of PIN1 or other PIN1 containing complexes.

Furthermore, the structure coordinates of the PIN1 PPIase polypeptides, described herein, are useful in solving the structure of other PIN1 proteins that have amino acid substitutions, additions and/or deletions. These PIN1 mutants may optionally be crystallized in complex with a chemical entity, such as Compound I. The crystal structure of such a complex may then be solved by molecular replacement and compared with structure of the PIN1 PPIase polypeptides described. Potential sites for modification within the various binding sites of the enzyme may thus be identified. This information provides an additional tool for determining the efficient binding interactions, for example, increased hydrophobic interactions, between PIN1 PPIase and a chemical entity.

The structure coordinates are also useful to solve the structure of crystals of PIN1 or PIN1 homologues complexed with chemical entities. This approach enables the determination of the important sites for interaction between chemical entities, including potential PIN1 modulators with the PIN1 substrate-binding site. For example, high resolution X-ray diffraction data collected from crystals exposed to different types of solvent allows the determination of where each type of solvent molecule resides. Small molecules that bind tightly to those sites can then be designed and synthesized and tested for their ability to modulate PIN1 PPIase activity.

All of the complexes referred to above may be studied using known X-ray diffraction techniques and may be refined versus 1.5-3.0 Å resolution X-ray data to an R value of about 0.20 or less using computer software, such as X-PLOR (Brunger, 1992, supra, distributed by Accelrys, Inc., San Diego, Calif. This information may be used to optimize known PIN1 PPIase modulators, and to design new PIN1 PPIase modulators.

E. Peptidyl-Prolyl Isomerase Assay

PIN1 is a phosphorylation dependent peptidyl-prolyl isomerase. Peptidyl-prolyl ismomerase activity for the peptides of the invention can be measured using a spectrophotometric assay based on the coupled chymotrypsin catalyzed, cis-trans conformation dependent cleavage of a para-nitroanaline-containing peptide substrate. This rotamase assay is described by Kofron et al. (Biochemistry 30, 6217-6134 (1991)) and its application to PIN1 isomerase activity is described by Yaffe et al. (Science 278, 1957-1960 (1997)). Cleavage of the isomerized peptide releases para-nitroanaline, which can be monitored by an increase in absorbance at 390 nm. The PIN1 peptide substrate, succinyl-alanine-leucine-proline-phenylalanine-paranitroaniline (Suc-AEPF-pNA) (Bachem California, Inc., Torrence, Calif.) is kept in a predominantly cis conformation with an anhydrous TFE (trifluorethanol)/LiCl (lithium chloride) solvent mixture. Upon dilution into an aqueous assay mixture containing peptides with PIN1 PPIase activity, the peptide substrate undergoes PIN1 catalyzed isomerization to the trans conformation. Chymotrypsin or other suitable protease, such as Subtilisin Carlsberg cleaves the trans product to form free para-nitroanaline. To minimize the spontaneous isomerization of the peptide substrate, reactions are performed at 15° C. Using this method, both the wild-type PIN1 and mutant PIN1 PPIase (K77Q/K82Q) (SEQ ID NO:4) at a concentration of 0.033 nM with 100 μM Suc-AEPF-pNA had a rate of 0.2. The Kᵢ of Compound I (Example 1) and K77Q/K82Q was 0.06 μM.

The following examples are for the purpose of illustrating various embodiments and features of the invention

### EXAMPLES

### Example 1

### Synthesis of a PIN1 Inhibitor-compound I

Compound I was synthesized according to scheme 1. The abbreviations employed in Scheme I have the following meaning unless otherwise indicated: CBzCl=Benzyl chloroformate; MCPBA=3-chloroperoxybenzoic acid; Pd:palladium; ETOH=ethyl alcohol; EtOAc=ethyl acetate; Ph=phenyl; and Bn=benzyl.

Synthesis of Compound I-Scheme 1:

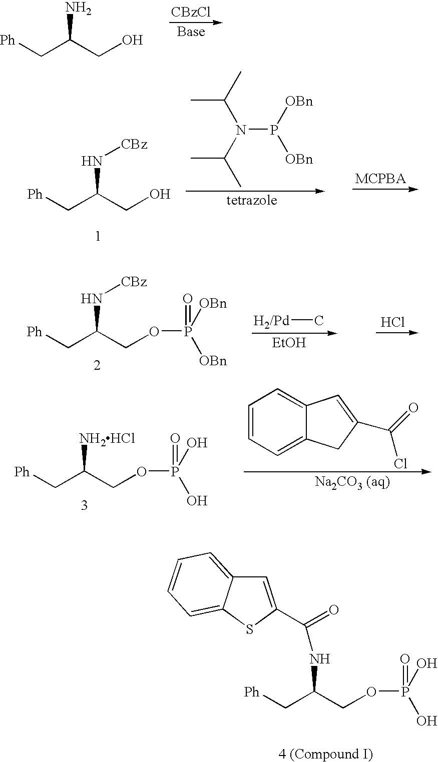

### Example 1A

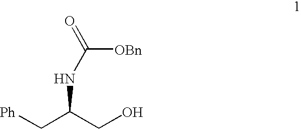

Alcohol 1: To a methylene chloride solution (80 mL) of D-phenylalaninol (1.15 g, 7.61 mmol) was added triethylamine (1.59 mL, 11.4 mmol) and benzyl chloroformate (1.19 mL, 8.37 mmol). The mixture was stirred for 3 hours (h) and then concentrated. The residue was dissolved in methylene chloride (50 mL) and washed with brine (1×50 mL). The solution was dried (Na₂SO₄) and concentrated. After column chromatography purification (10 to 30% EtOAc in hexanes), the title compound was obtained in 73% yield (1.59 g).

¹H NMR (CDCl₃): δ 7.46-7.15 (10H, m), 5.11 (2H, s), 4.96 (1H, m), 3.98 (1H, m), 3.72 (1H, m), 3.63 (1H, m), 2.89 (1H, d, J=7.2 Hz).

MS (ESP): 286 (M+H⁺); 284 (M−H)^{—}.

### Example 1B

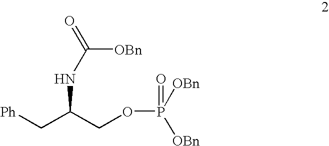

Phosphate Benzyl Ester 2: To an acetonitrile solution (40 mL) of the alcohol 1 (1.58 g, 5.54 mmol) and 1H-tetrazole (1.05 g, 15 mmol) was added dibenzyl N,N-diisopropylphosphoramidite (3.72 mL, 11.1 mmol) at 25° C. After 3h, MCPBA (4.19 g, 70% pure, 13.85 mmol) was added to the suspension. The solution was diluted with EtOAc (100 mL), washed with concentrated NaHSO₃ solution (2×80 mL), dried over MgSO₄ and concentrated in vacuo. The residue was purified by column chromatography (10-30% EtOAc in hexanes) to give 2.88 g of the title compound in 95% yield.

¹H NMR (CDCl₃): δ 7.47-7.05 (20H, m), 5.19-4.96 (7H, m), 4.09-3.83 (3H, m), 2.93-2.67 (2H, m).

MS (positive ESP): 568 (M+Na⁺); MS (negative ESP): 580 (M+Cl)^{—}.

### Example 1C

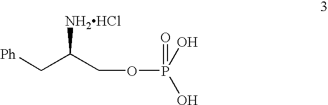

(2R)-2-amino-3-phenylpropyl-dihydrogen-phosphate-hydrochloride (3): To an ethanol solution of the phosphate benzyl ester (2, 2.88 g, 5.28 mmol) was added palladium on carbon (10%, 300 mg). The suspension was kept under hydrogen atmosphere (1 atm) for 4 h, and was then filtered through a pad of Celite. The collected solid was washed with methylene chloride. The mixture of the solid and Celite was suspended in 5% HCl solution and stirred for 20 min. After filtration, the filtrate was concentrated to dryness, affording 1.2 g of the title compound in 86% yield.

¹H NMR (CD₃OD): δ 7.49-7.25 (5H, m), 4.22-4.08 (11H, m), 4.0 (1H, m), 3.72 (1H, m), 3.03 (2H, d, J=7.5 Hz).

LCMS: 232 (M+H⁺); 230 (M−H)^{—}.

HRMS (MALDI) calc for C₉H₁₅NO₄P (M+H⁺) 232.0733; found 232.0736.

### Example 1D

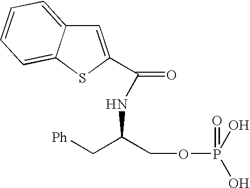

Compound I-Phosphoric acid mono-{(R)-2-[(1-benzo[b]thiophen-2-yl-methanoyl)-amino]-3-phenyl-propyl} ester (4): To a sodium carbonate solution (1M, 1 mL) was added the aminophosphate 3 (48 mg, 0.179 mmol) and benzothiophene-2-carbonyl chloride (35 mg, 0.179 mmol). After 15 h, it was acidified to pH˜1 by addition of concentrated HCl solution at 0° C. Preparative HPLC purification gave 34 mg (48% yield) of the title compound.

¹H NMR (CD₃OD): δ 7.96 (1H, s), 7.90 (2H, m), 7.43 (2H, m), 7.37-7.17 (5H, m), 4.50 (1H, m), 4.10 (2H, m), 3.09 (1H, dd, J=13.9, 6.6 Hz), 3.00 (1H, dd, J=13.9, 7.8 Hz).

HRMS (MALDI) calc for C₁₈H₁₈NO₅PSNa (M+Na⁺) 414.0540; found 414.0536.

### Example 2

### Cloning and Biochemical Analysis of PIN1 PPIase Polypeptides

The PPIase domain from wild-type PIN1 was amplified by PCR (Mullis et al., CSH Symp. Quantum Biol. 51:263-273 (1986); Saiki et al., Science 239:487-491 (1988)), using a pET3a vector (Novagen, Madison, Wis.) containing the coding sequence for full-length PIN1. The primers used were as follows:

| | |
|---|---|
| Forward primer-5′ AGCAGCCATATGGGCAAAAACGGGCAGGGGGAGCCT-3′ | (SEQ ID NO: 5) |
| Reverse primer-5′-CTTGGATCCTCACTCAGTGCGGAGGATGAT-3′ | (SEQ ID NO: 6) |

The amplified DNA was cloned into the NdeI and BamHI sites of the bacterial expression vectors pET3a and pET28a (Novagen), and sequence verified. pET28a contains a 6 Histidine tag followed by a thrombin cleavage site.

The amino acid sequence of the PIN1 PPIase domain corresponds to amino acids 45-163 of full-length PIN1 (GenBank Accession No. XM_{—}009024) and is shown below:

| | |
|---|---|
| 45 GKNGQG EPARVRCSHL LVKHSQSRRP SSWRQEKITR TKEEALELIN | (SEQ ID NO: 7) |
| GYIQKIKSGE EDFESLASQF SDCSSAKARG DLGAFSRGQM QKPFEDASFA | |
| LRTGEMSGPV FTDSGIHIIL RTE 163 | |

The pET3a vector coded for a recombinant PIN1 PPIase polypeptide, which contained an additional M residue at the N-terminus. The pET28a vector expressed a recombinant PIN1 PPIase polypeptide, which upon thrombin cleavage, generated a polypeptide with four additional amino acids at the N-terminus corresponding to the following amino acid sequence: 5′-GSHM-3′.

### Example 3

### PIN1 PPIase K77Q/K82Q

The double mutant, K77Q/K82Q, which contains the amino acid lysine instead of the amino acid glutamine at positions 77 and 88, was generated by the QuickChange™ site-directed mutagenesis method (Stratagene, La Jolla, Calif.) following the manufacturer's protocol and as described below (Catalog # 200518; revision # 108005h), using the pET28a PPIase vector and the following PCR primers:

| | |
|---|---|
| PIN1K77/82Q Forward: | |
| 5′-GCGGCAGGAGCAGATCACCCGGACCCAGGAGGAGGCCCTGGAGC-3′ | (SEQ ID NO: 8) |
| PIN1K77/82Q Reverse: | |
| 5′-GCTCCAGGGCCTCCTCCTGGGTCCGGGTGATCTGCTCCTGCCGC-3′ | (SEQ ID NO: 9) |

Mutagenesis Protocol:

A sample reaction mixture was prepared by combining 5 μl of 10× reaction buffer (100 mM KCl, 100 mM (NH₄)₂SO₄, 200 mM Tris-HCl (pH 8.8), 20 mM MgSO4, 1% Triton® X-100, and 1 mg/ml nuclease-free bovine serum albumin (BSA)); 5-50 ng of dsDNA template; 125 ng of each primer; 1 μl of dNTP mix; ddH₂O to a final volume of 50 μl.

To the sample reaction mixture was added 1 μl of PfuTurbo® DNA polymerase (2.5 U/μl). The reactions were overlayed with 30 μl of mineral oil. Each reaction was cycled using the following cycling parameters: Segment 1—one cycle at 95° C. for 30 seconds; Segment 2-12 to 18 cycles at 95° C. for 30 seconds, 55° C. for one minute and 68° C. for 2 minutes/kb of plasmid length. After cycling, 1 μl of Dpn1 restriction enzyme (10 U/μl) was added below the mineral oil overlay. The reaction mixtures were gently and thoroughly mixed and spun down in a microcentrifuge for 1 minute. After centrifugation, the reactions were incubated at 37° C. for 1 hour to digest the parental supercoiled dsDNA. One μl of the Dpn1-treated DNA from each control and sample reaction were used to transform E. coli strain DH5α.

The K77Q/K82Q PIN1 PPIase mutant was sequence verified.

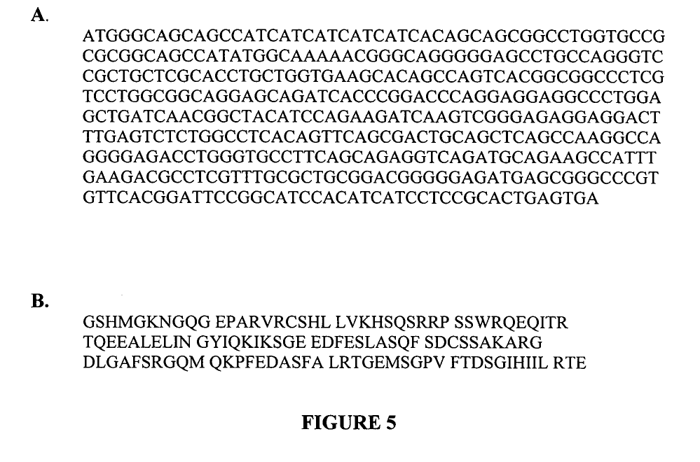

The amino acid sequence of the K77Q/K82Q PIN1 PPIase mutant is shown in FIG. 5. The amino acid sequence of the PPIase domain of the K77Q, K82Q PIN1 mutant is shown below.

| | |
|---|---|
| 45 GKNGQG EPARVRCSHL LVKHSQSRRP SSWRQEQITR TQEEALELIN | (SEQ ID NO: 10) |
| GYIQKIKSGE EDFESLASQF SDCSSAKARG DLGAFSRGQM QKPFEDASFA | |
| LRTGEMSGPV FTDSGIHIIL RTE. | |

### Example 4

### Purification and Biochemical Analysis of PIN1 PPIase Polypeptides

A. Fementation

E. coli BL21(DE3) cells containing a PET28a vector encoding for either wild-type PIN1 PPIase or mutant PPIase K77Q/K82Q were inoculated into 5 ml of 2×YT media (per liter: 16 g tryptone, 10 g yeast extract, 5 g NaCl) containing 50 μg/ml Kanamycin in a Falcon 2059 tube. This culture was shaken overnight at 250 rpm at 37° C. The overnight culture was diluted 100-fold in 2×YT medium containing 50 μg/ml kanamycin. The diluted culture was shaken at 250 rpm at 37° C. to an OD₅₉₅ of from 0.6 to 0.8. 0.3 mM IPTG was added and the culture shaken overnight at 250 rpm at 25° C. The overnight cell culture was centrifuged at 5000 rpm for 20 min. The pellets were resuspended in 10× buffer A (50 mM Na₃PO₄, pH 7.5, 0.5 M NaCl, 20 mM imidazole, 5 mM 2-mercaptoethanol). The suspension was passed through a high-pressure microfluidizer. The homogenate was centrifuged down in a Beckman ultracentrifuge at 40,000 rpm at 4° C. for 45 min. The clear supernatant was saved for further purification.

B. Purification

The clarified supernatant was loaded onto a Ni-NTA column (20 ml) at 4 ml/min. The column was washed with 200 ml of buffer A. A linear gradient (400 ml) was run at 4 mmin from 100% buffer A to 100% buffer B (50 mM Na₃PO₄, pH 7.5, 0.5 M NaCl, 500 mM imidazole, 5 mM 2-mercaptoethanol). The fractions were collected (6 ml) and separated using SDS-PAGE (12%). The fractions containing 6×His PIN1 PPIase were collected and pooled. The pooled fractions were dialyzed against 4 liters of buffer C (25 mM HEPES pH 7.5, 100 mM NaCl, 5 mM 2-mercaptoethanol) overnight at 4° C.

C. Thrombin Cleavage

To the pooled fractions containing 6×His PIN1 PPIase was added biotinylated thrombin (1 unit per 10 mg protein). The solution was gently rotated overnight at 4° C. The overnight solution was passed through a Ni-NTA column (5 ml) and a Streptavidin-Agarose column (1 ml). The flowthrough was collected and concentrated to about 10 mg/ml for further studies.

D. PIN1 Peptidyl-Prolyl Isomerase Assay

Peptidyl-prolyl isomerase reactions were carried out in 25 mM MOPS [3-(N-Morpholino)propanesufonic acid], pH 7.5, 0.5 mM TCEP [Tris(2-carboxyethyl)phosphine hydrochloride], 2% DMSO, 5 μl of a 25 mg/ml solution of Subtilisin Carlsberg Protease (Sigma), 50 nM PIN1-PPIase, and 100 μM Suc-AEPF-pNA peptide substrate. Reactions were cooled to 15° C. and initiated with the addition of Suc-AEPF-pNA. The absorbance at 390 nm was monitored continuously until all substrate had been converted to the cleaved product. This data, the progress curve, was then fitted to an exponential equation to determine a rate constant k for the reaction. The rate constant k is linearly proportional to the concentration of active enzyme present in the assay mixture once the rate constant for the spontaneous isomerization is subtracted. The Kₘ for this substrate was much higher than 100 μM ([S]<<Kₘ). Therefore, during the inhibition experiment, the IC₅₀ for this non-tight-binding inhibitor was essentially Kᵢ. Without an inhibitor present, both wild type human PIN1 and mutant PIN1 PPIAse, at 0.033 nM with 100 μM Suc-AEPF-pNA, had a rate of 0.2. The Kᵢ of Compound I and mutant PPIase K77Q/K82Q was 0.06 μM.

E. Isothermal Titration Calorimetry

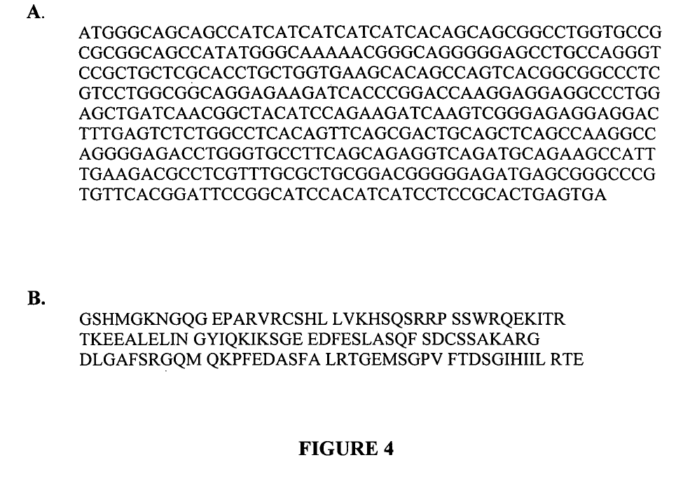

The binding of Compound I to a His-tagged construct of the K77Q/K82Q PPIase domain (FIG. 4) was studied by isothermal titration calorimetry (ITC) as follows. The titrations were performed in duplicate and the stated uncertainties are the standard deviations of the averaged results.

Following a preliminary 2 μL injection, twenty to twenty-five 10 μL injections of a 200 μM solution of the PPIase polypeptide was titrated into a 10 μM solution of Compound I. The titrations were performed using a VP-ITC (MicroCal, Northampton, Mass.) at 15.0° C. with stirring set at 270 rpm, 4 minutes injection intervals, and a 20 second injection duration for the 10 μL injections. The working volume of the ITC cell was 1.414 mL. Both solutions contained 25 mM MOPS pH 7.5, 0.5 mM TCEP and 2.0% DMSO (vol./vol.). The PPIase polypeptide solution was prepared by exhaustively dialyzing a stock protein solution against several changes of dialysis buffer (25 mM MOPS pH 7.5, 0.5 mM TCEP) at 4.0° C.

After dialysis the protein was centrifuged to remove any particulate matter. The protein concentration was then determined by absorbance using an extinction coefficient that had been calculated based on the tryptophan and tyrosine content of the protein. The dialysed protein was then diluted with the dialysate and 2.0% (volume to volume) DMSO was added to yield a final concentration of 200 μM protein. A 20 mM Compound I stock solution was prepared by dissolving a small amount of the compound in DMSO. An aliquot of the stock solution was diluted in DMSO and then an appropriate volume of dialysate was added. The final DMSO concentration was 2.0% (volume to volume) and the final compound concentration was 10 μM.

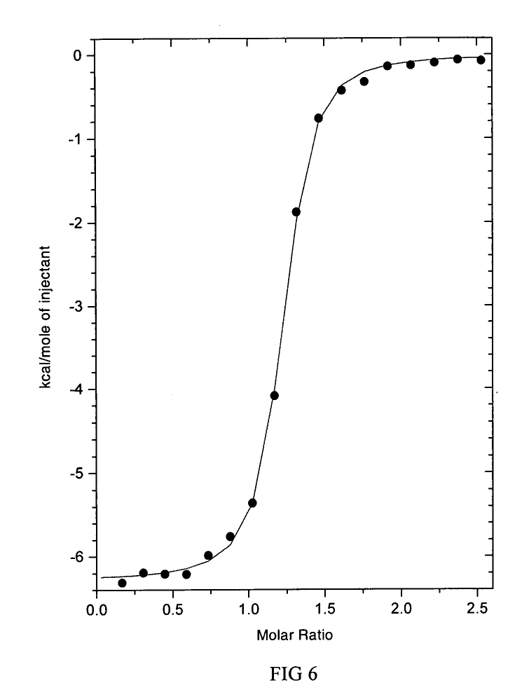

Appropriate control titrations (buffer into buffer, buffer into compound, and protein into buffer) were performed to determine the heats of dilution. Prior to fitting for the binding parameters, the observed heats of binding were corrected for heat of dilution of the protein. The machine blank correction (buffer into buffer) and the heat of dilution of the compound were comparable and as such were neglected when correcting for the heats of dilution. The data were fit using the ORIGIN® software package (MicroCal) provided with the ITC (FIG. 6). In FIG. 6, the solid line represents the best fit of the corrected binding data using the ORIGIN software package (ka=1.42×10⁷ M⁻¹, C value=142). The One Set of Sites model with ligand in the cell was selected. The lower than one to one stoichiometry that was observed is most likely the result of the presence of a small amount of inactive enzyme in the stock protein sample. This result was consistent with the observation of a slight reduction in the enzymatic activity of the protein sample.

The stoichiometry, dissociation constant and enthalpy of binding were determined to be 0.854 (±0.003), 67 (±5) nM and −7.3 (±0.1) kcal/mol, respectively.

### Example 5

### Crystallization of PPIase Polypeptides and PPIase PPIase/Compound I Complex

Crystals of the apoenzyme (thrombin cut PPIase K77Q/K82Q) were grown at 13° C. via the hanging-drop vapor-diffusion method. Crystals were obtained by mixing equal volumes of protein solution (10-15 mg/ml protein) and reservoir solution of 1.2-1.4 M Na Citrate, with 0.1 M Hepes (or Borate, when pH>8.5) at a pH range of 7.5-10.0 (optimum pH=8.8), and 5 mM DTT. Crystals typically grew within 3 days. For X-ray data collection, crystals were transferred into a cryoprotectant containing 20% glycerol in addition to the reservoir solution and flash frozen in liquid nitrogen. The crystals, which were determined to belong to the monoclinic space group C2 with a=116.84, b=35.82, c=51.40 Å alpha=90.0, beta=100.33, and gamma=90.0 degrees, contained two molecules per asymmetric unit.

Crystals of thrombin cut PPIase K77Q/K82Q and Compound I were obtained by crystallization under conditions similar to those described above for the apoenzyme. The protein was diluted to 10 mg/ml, then exposed to Compound I (dissolved in 100% DMSO) by adding to a final concentration of 1 mM. The ratio of PPIase polypeptide to Compound I was 1:5. The reservoir solution contained 1.4 M Na citrate, with 0.1 M Hepes at pH 7.5 (titrated with HCl) and 10 mM DTT. The resulting protein/Compound I solution was then incubated for 24 hours at 4° C., and filtered through a 0.45 μM cellulose-acetate membrane prior to setting up crystallization experiments. Crystals grew within 3 days. The crystal:ligand complexes had the identical space group (C2) and similar cell dimensions as described above for the apoenzyme.

### Example 6

### PPIase K77Q/K82Q Structure Solution

The structure of the PPIase mutant K77Q/K82Q was solved by molecular replacement (MR) using EPMR software (Kissinger et al., Acta Cryst. D55:484-491 (1999)), with residues 55-163 of the native PIN1 structure as the MR probe. The R-factor for the correctly positioned and oriented dimer was 39.7% for data in the 10-4.0 Å range. The MR solution was refined by ARP/wARP (EMBL) to an R-factor of 17.6% to produce a SIGMAA weighted 2Fo-Fc map for fitting. Refinement was carried out using simulated annealing and conjugate gradient minimization protocols in the program X-PLOR (Brunger, 1992, supra) (see Table I for refinement statistics). The final model included all atoms for residues 51-163 in molecule A (excluding the side chain atoms of residues 69 and 87), all atoms for residues 54-163 in molecule B (excluding the side chain atoms of residues 69, 94, and 95) plus 242 waters. The structure coordinates for the apoenzyme are given in Table 11.

### Example 7

### PPIase K77Q/K82Q Complexed with Compound I Structure Solution

Protein atomic coordinates from the crystal structure of PPIase K77Q/K82Q were used to initiate rigid-body refinement in X-PLOR followed by simulated annealing and conjugate gradient minimization protocols. Placement of the inhibitor and addition of ordered solvent into difference electron density maps was followed by subsequent rounds of refinement using X-PLOR (see Table I for refinement statistics). The final model included all atoms for residues 51-163 in molecule A (excluding the side-chain atoms of residue 87), all atoms for residues 54-163 in molecule B (excluding the side-chain atoms of residues 94 and 95) plus Compound I and 181 waters. Inhibitor occupancy in molecule B was lower than that observed for molecule A.

The results from the crystallographic analysis are shown in Table I below. Crystal structure coordinates are set forth in Table III.

Table I. Statistics for Crystallographic Analysis

PPIase(K77/K82Q)
PPIase(K77/K82Q) + Compound I

| | | |
|---|---|---|
| Resolution (Å) | 1.85 | 2.00 |
| Reflections measured | 50117 | 65503 |
| Unique reflections | 16272 | 14274 |
| Completeness (%) | 89.5(53.4) | 97.9 |
| R¹_{sym} | 4.3(12.6) | 5.8(17.1) |
| R_{cryst}² (%) | 20.9 | 20.3 |

¹R_{syn} = Σ|I(\overline{h}) − I(h)ᵢ|/ΣI(h)ᵢ * 100
h, i = 1
h, i = 1
Where I(h)ᵢ is the i^{th} measurement of reflection h and I(\overline{h}) is the mean value of the N equivalent reflections
²R_{cryst} = Σ||F_{obs}| − |F_{calc}||/Σ|F_{obs}|
Where summation is over data used in the refinement

### Example 8

### High-throughput Assay Utilizing Peptides that Contain the PIN1 PPIase Domain

This assay is based on fluorescence polarization. In fluorescence polarization detection, monochromatic light passes through a polarized filter and excites molecules in the sample well. Only those molecules that are oriented properly in the polarized plane absorb light, become excited, and subsequently emit light. The emitted light is detected after passing through polarizing filters that are oriented parallel and perpendicular to the plane of excitation. Since small molecules rotate more quickly than large molecules (e.g. in the form of a bound complex), the parallel (S) and perpendicular (P) measurements are closer and the difference is lower. Fluorescence polarization is measured in mP (milliP) which is defined using the following equation:

mP=1000*(S−P)/(S+P)

For the PIN1 assay, library compounds compete with fluorescein-tagged Pintide to bind the PPIase domain of PIN1. After a short incubation, samples are assayed using fluorescence polarization. The excitation and emission of fluorescein occur at 485 nm and 530 nm, repectively. The assay is homogeneous and performed with or without the presence of library compounds. Formation of a complex between fluorescein-tagged Pintide and the PPIase domain of PIN1 leads to large differences between the S and P measurements, resulting in high mP values. Compounds that bind to the PPIase domain of PIN1 and prevent the formation of this complex lower the mP values.

Materials and Reagents

Experiments were performed in either 96-well plates or 384-well black flat bottom polystyrene non-binding surface (NBS) plates (Costar). The PPIase substrate was a fluorescein-tagged Pintide, FL-WFYpSPFLE (SEQ ID NO:11) where pS equals phosphorylated serine. The inhibitor control was Pintide without the fluorescein tag. Fluorescent Pintide was either purchased (AnaSpec, Inc., San Jose, Calif.) or synthesized as described herein. The buffer conditions were 25 mM MOPS [3-(N-Morpholino)propanesufonic acid], and 0.5 mM TCEP [Tris(2-carboxyethyl)phosphine hydrochloride], at pH 7.5. For inhibitor controls, free Pintide was used at 50, 10, and 2 μM (IC₅₀ of free Pintide is about 7-10 μM). Excitation was measured at 485 nm and emission was measured at 530 nm. Readings were taken in a Florescence Polarization reader (Molecular Devices Analyst).

Pintide Synthesis

Pintide (WFYpSPFLE) was synthesized on an Applied Biosystems 433A Peptide Synthesizer on a 0.1 mmol scale using standard Fmoc chemistry and preloaded HMP resin. After thorough washing with dichloromethane (DCM) (Fisher), the peptide was cleaved from the resin and deprotected in trifluoroacetic acid (TFA) (Aldrich) with ethanedithiol and thioanisole present as scavengers. The solution was filtered into cold m-tert butyl ether (MTBE) (Aldrich) to precipitate the peptide and centrifuged at 6 Krpm for 3 minutes. The resulting pellet was washed and centrifuged in cold MTBE four times then dried under vacuum. The dried precipitate was resuspended and lyophilized overnight.

Purification was performed on an ISCO 2350 HPLC with a Linear LS500 scanning detector and a Foxy II fraction collector. The purification conditions were as follows: mobile phase was 0.1% TFA:H₂O and eluent was 0.1% TFA:CH₃CN (acetonitrile (Omnisolve, VWR)); the gradient was 5% to 95% in 30 minutes on a 25×1 cm Hypersil ODS (5 μm, 300A, Phenomenex); the flow rate was 2.5 mmin; and fractions at 30-second intervals.

Fractions were analyzed on an HP 1050 with the same buffer system and gradient on a 100×4.6 mm Hypersil ODS column (Hewlett-Packard). Pure product (Pintide; elution time=12.22 minutes) was lyophilized overnight. Compound identity was confirmed by MALDI-TOF mass spectroscopy.

Fluorescein modification was carried out following the basic protocol published by Molecular Probes (MP-00143; Aug. 19, 1998) as described below.

Twenty mg of purified, lyophilized Pintide (peptide content ˜60% so, 12 mg actual peptide) was resuspended in 1.75 ml of 0.1M NaHCO₃ (sodium bicarbonate) (Sigma), pH 8.3. 3.3 mg fluorescein-5-EX succinimidyl ester (Molecular Probes #F-6130) was resuspended in DMSO at 10 mg/ml (330 μl). 165 μl of this solution was added dropwise to the peptide solution under continuous stirring at room temperature. After 30 minutes, the remaining 165 μl was added dropwise under continuous stirring. After 60 minutes, the solution was loaded on the HPLC (under conditions described previously) to stop the reaction and facilitate purification. Fractions were analyzed as previously described and the product (fluroescein-tagged Pintide; elution time=14.58 minutes) was lyophilized overnight. Compound identity was confirmed byMALDI-TOF mass spectroscopy.

Assay Plate Format and Screening Conditions for 96-well Plates:

Forty-five μL of assay buffer containing 20 μM fluorescein-Pintide was dispensed into each of the wells. Test compounds (1 μL of a 0.5 mM stock concentration in DMSO) were added to columns 1-22. The 6His-PPIase domain of PIN1 (5 μL of a 4 μM solution in assay buffer) was added to all wells in columns 1-22 and most wells in columns 23-24. The following controls were used in columns 23-24: wells A23-F24 were DMSO controls and were used to calculate the maximum value; wells G23-H24, 123-J24, and K23-L24 were inhibitor controls at 50 μM, 10 μM, and 2 μM free Pintide, respectively; and wells M23-P24 contained no PPIase and were used to calculate the minimum value. The assay was incubated at room temperature for 10 minutes and immediately read at excitation 485 nm and emission 530 nm in fluorescence polarization mode. The percent inhibition of each well was calculated using the following equation:

% inhibition=100*(1−(mP_{well}−Min_{average})/(Max_{average}−Min_{average}))

The order of addition can be changed. For example, in a variation of the present assay, compounds can be added to the plate first, followed by fluorescein-Pintide in asssay buffer, and finally 6His-PPIase. As currently designed, the assay is a competition assay.

The premise of the assay is different when the fluorescein-Pintide and 6His-PPIase are added first followed by compound addition. The fluorescein-Pintide and 6His-PPIase preform a complex. When the compound is added, it must displace the fluorescein-Pintide from the binding site. This may occur depending on the K_{D} of the compound; however, a longer incubation is required.

The foregoing description has been provided to illustrate the invention and its preferred embodiments. The invention is intended not to be limited by the foregoing description, but to be defined by the appended claims.

What is claimed is:

1. An isolated polynucleotide encoding a polypeptide comprising a PIN1 PPIase that does not contain a WW domain.

2. An isolated polynucleotide that:
(a) encodes a polypeptide comprising the amino acid sequence of SEQ ID NO:2; and
(b) does not encode a WW domain.

3. An isolated polynucleotide comprising the polynucleotide sequence of SEQ ID NO:1, wherein said polynucleotide does not encode for a WW domain.

4. An isolated polypeptide comprising the amino acid sequence of SEQ ID NO:2, wherein said polypeptide does not contain a WW domain.

5. An isolated polynucleotide that
(a) encodes a polypeptide comprising the amino acid sequence of SEQ ID NO:4; and
(b) does not encode a WW domain.

6. An isolated polynucleotide comprising the polynucleotide sequence of SEQ ID NO:3, wherein said polynucleotide does not encode a WW domain.

7. An isolated polypeptide comprising the amino acid sequence of SEQ ID NO:4, wherein said polypeptide does not contain a WW domain.

8. A polynucleotide according to claim 2, further comprising at least one polynucleotide sequence that encodes a proteolytic cleavage site.

9. A polynucleotide according to claim 5, further comprising at least one polynucleotide sequence that encodes a proteolytic cleavage site.

10. A polynucleotide according to claim 8, wherein the proteolytic cleavage site is a thrombin cleavage site.

11. A polynucleotide according to claim 9, wherein the proteolytic cleavage site is a thrombin cleavage site.

12. A polynucleotide according to claim 2, further comprising at least one polynucleotide sequence that encodes a histidine tag.

13. A polynucleotide according to claim 5, further comprising at least one polynucleotide sequence that encodes a histidine tag.

14. An isolated polypeptide encoded by the polynucleotide of claim 1.

15. An isolated polypeptide encoded by the polynucleotide of claim 6.

16. An isolated polypeptide encoded by the polynucleotide of claim 7.

17. A vector comprising the polynucleotide of claim 1.

18. A vector according to claim 17, wherein said vector is an expression vector comprising the polynucleotide of claim 1 operably linked to a promoter.

19. A eukaryotic cell line or prokaryotic cell transformed or transfected with the vector of claim 17.

20. A eukaryotic cell line or prokaryotic cell transformed or transfected with a polynucleotide comprising the polynucleotide of claim 1.

21. A method of producing a polypeptide or fragment thereof comprising culturing the cell line or cell of claim 19 under conditions such that said polypeptide is expressed, and recovering said polypeptide.

22. A method of assaying a compound for its PIN1 modulating ability comprising:
(a) adding a test compound to a polypeptide comprising a PIN1 peptidyl-prolyl isomerase, wherein said polypeptide does not contain a WW domain;
(b) measuring said polypeptide's peptidyl-prolyl isomerase activity; and
(c) determining if the activity of the polypeptide is modulated by said test compound.

23. A method according to claim 22, wherein said polypeptide is encoded by a polynucleotide comprising the polynucleotide of claim 2 or 5.

24. A method according to claim 22, wherein said method is done in a high-throughput format.

25. A crystal structure comprising a PIN1 peptidyl-prolyl isomerase (PPIase) polypeptide that does not contain a WW domain.

26. A crystal structure comprising the polypeptide encoded by the polynucleotide of claim 2, or a fragment thereof.

27. A crystal structure comprising the polypeptide encoded by the polynucleotide of claim 5, or a fragment thereof.

28. A crystal structure according to claim 25, wherein said crystal structure diffracts X-rays at a resolution value greater than or equal to 3 Å.

29. A crystal structure according to claim 25, wherein said crystal structure diffracts X-rays at a resolution value of greater than or equal to 2 Å.

30. A crystal structure comprising a PIN1 PPIase polypeptide:ligand complex, wherein said polypeptide does not contain a WW domain.

31. A crystal structure according to claim 30, wherein said polypeptide is encoded by the polynucleotide sequence of claim 2 or 5.

32. A crystal structure according to claim 30, wherein said crystal structure diffracts X-rays at a resolution of greater than or equal to 3.0 Å.

33. A crystal structure according to claim 25, wherein said PIN1 peptidyl-prolyl isomerase polypeptide has a three-dimensional structure characterized by the structure coordinates of Table II.

34. A crystal structure according to claim 30, wherein said ligand is a modulator of PIN1 peptidyl-prolyl isomerase activity.

35. A crystal structure according to claim 34, wherein said modulator of PIN1 peptidyl-prolyl isomerase activity is a compound of the formula:

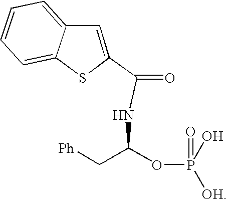

36. A crystal structure according to claim 30, wherein said PIN1 PPIase polypeptide has a three-dimensional structure characterized by the structure coordinates of Table III.

37. A method of using a three-dimensional structure of a complex comprising a PIN1 peptidyl-prolyl isomerase polypeptide devoid of the WW domain and compound I, as defined by the structure coordinates of Table III or a portion thereof, in a drug discovery strategy comprising:
(a) selecting a potential drug using computer-aided drug design with the three-dimensional structure determined from one or more sets of atomic coordinates in Table III, wherein said selecting is performed in conjunction with computer modeling;
(b) contacting said potential drug with a polypeptide containing a functional PIN1 peptidyl-prolyl isomerase; and
(c) detecting the binding of said potential drug with said polypeptide.

38. A method of using a three-dimensional structure of a complex comprising a PIN1 peptidyl-prolyl isomerase polypeptide devoid of the WW domain and compound I and as defined by the structure coordinates of Table III, or a portion thereof, in a drug discovery strategy comprising:
(a) selecting a potential drug using computer-aided drug design with the three-dimensional structure determined from one or more sets of structure coordinates in Table III, wherein said selecting is performed in conjunction with computer modeling;
(b) contacting said potential drug with a polypeptide containing a functional PIN1 peptidyl-prolyl isomerase; and
(c) determining if said potential drug modulates the peptidyl-prolyl isomerase activity of a polypeptide containing a PIN1 peptidyl-prolyl isomerase.

39. A method for evaluating the potential of a chemical entity to associate with a molecule or molecular complex comprising a binding pocket defined by a set of structure coordinates comprising structure coordinates of PIN1 PPIase amino acids His59, Leu61, Lys63, Ser67, Arg68, Arg69, Cys113, Leu122, Met130, Gln131, Phe134, Glu135, Thr152, Ser154, and His157, according to Table III, or a portion thereof, comprising the steps of:
(a) employing computational means to perform a fitting operation between the chemical entity and a binding pocket defined by structure coordinates of PIN1 PPIase amino acids His59, Leu61, Lys63, Ser67, Arg68, Arg69, Cys113, Leu122, Met130, Gln131, Phe134, Gli135, Thr152, Ser154, and His157, according to Table III; and
(b) analyzing the results of said fitting operation to quantify the association between the chemical entity and the binding pocket.

40. A method according to claim 39, wherein said set of structure coordinates comprises structure coordinates of PIN1 PPIase amino acids Arg54, Arg56, His59, Leu61, Lys63, Ser67, Arg68, Arg69, Ser72, Trp73, Ser111, Asp112, Cys113, Ser114, Ser115, Ala116, Lys117, Ala118, Arg119, Gly120, Asp121, Leu122, Gly123, Ala124, Phe125, Ser126, Arg127, Gly128, Gln129, Met130, Gln131, Lys132, Pro133, Phe134, Glu135, Thr152, Asp153, Ser154, and His157 according to Table III.

41. A method according to claim 39, wherein said method evaluates the potential of a chemical entity to associate with a molecule or molecular complex defined by structure coordinates of substantially all of the PIN1 PPIase amino acids, as set forth in Table III.

42. A method for identifying a modulator of a molecule comprising a PIN1 PPIase substrate-binding domain comprising the steps of:
(a) using a set of structure coordinates comprising structure coordinates of PIN1 PPIase amino acids His59, Leu61, Lys63, Ser67, Arg68, Arg69, Cys113, Leu122, Met130, Gln131, Phe134, Glu135, Thr152, Ser154, and His157, according to Table III to generate a three-dimensional structure of a molecule comprising a PIN1 PPIase or PPIase-like substrate-binding pocket;
(b) employing said three-dimensional structure to design or select said modulator;
(c) synthesizing or obtaining said modulator; and
(d) contacting said modulator with said molecule to determine the ability of said modulator to interact with said molecule.

43. A method according to claim 42, wherein said set of structure coordinates used in step (a) comprises PIN1 PPIase amino acids Arg54, Arg56, His59, Leu61, Lys63, Ser67, Arg68, Arg69, Ser72, Trp73, Ser111, Asp112, Cys113, Ser114, Ser115, Ala116, Lys117, Ala118, Arg119, Gly120, Asp121, Leu122, Gly123, Ala124, Phe125, Ser126, Arg127, Gly128, Gln129, Met130, Gln131, Lys132, Pro133, Phe134, Glu135, Thr152, Asp153, Ser154, and His157 according to Table III.

44. A method according to claim 43, wherein the structure coordinates used in step (a) comprise substantially all the amino acids of PIN1 PPIase according to Table III.

45. A machine-readable medium having stored thereon data comprising the structure coordinates of a PIN1 PPIase substrate-binding site amino acids His59, Leu61, Lys63, Ser67, Arg68, Arg69, Cys113, Leu122, Met130, Gln131, Phe134, Glu135, Thr152, Ser154, and His157 according to Table III.

46. A machine-readable medium having stored thereon data comprising the structure coordinates of a PIN1 PPIase substrate-binding site comprising amino acids Arg54, Arg56, His59, Leu61, Lys63, Ser67, Arg68, Arg69, Ser72, Trp73, Ser111, Asp112, Cys113, Ser114, Ser115, Ala116, Lys117, Ala118, Arg119, Gly120, Asp121, Leu122, Gly123, Ala124, Phe125, Ser126, Arg127, Gly128, Gln129, Met130, Gln131, Lys132, Pro133, Phe134, Glu135, Thr152, Asp153, Ser154, and His157 according to Table III.

47. A machine-readable medium having stored thereon data comprising the structure coordinates of a PIN1 PPIase:Compound I complex according to Table III.

48. A method of obtaining structural information about a molecule or a molecular complex of unknown structure by using the structure coordinates set forth in Table III, comprising the steps of:
(a) generating X-ray diffraction data from said crystallized molecule or molecular complex; and
(b) applying at least a portion of the structure coordinates set forth in Table III to said X-ray diffraction pattern to generate a three-dimensional electron density map of at least a portion of the molecule or molecular complex.

49. A method for evaluating the ability of a compound to associate with a molecule or molecular complex comprising a PIN1 PPIase substrate-binding pocket, said method comprising the steps of:
(a) constructing a computer model of said binding pocket defined by a set of structure coordinates comprising structure coordinates of PIN1 PPIase amino acids His59, Leu61, Lys63, Ser67, Arg68, Arg69, Cys113, Leu122, Met130, Gln131, Phe134, Glu135, Thr152, Ser154, and His157 according to Table III;
(b) selecting a compound to be evaluated by a method selected from the group consisting of (i) assembling molecular fragments into said compound, (ii) selecting a compound from a small molecule database, (iii) de novo ligand design of said compound, and (iv) modifying a known modulator, or a portion thereof, of a peptidyl-prolyl isomerase;
(c) employing computational means to perform a fitting program operation between computer models of said compound to be evaluated and said binding pocket in order to provide an energy-minimized configuration of said compound in the binding pocket; and
(d) evaluating the results of said fitting operation to quantify the association between said compound and the binding pocket model, thereby evaluating the ability of said compound to associate with said binding pocket.

50. A method according to claim 49, wherein said binding pocket is defined by a set of structure coordinates comprising structure coordinates of PIN1 PPIase:compound I complex amino acids Arg54, Arg56, His59, Leu61, Lys63, Ser67, Arg68, Arg69, Ser72, Trp73, Ser111, Asp112, Cys113, Ser114, Ser115, Ala116, Lys117, Ala118, Arg119, Gly120, Asp121, Leu122, Gly123, Ala124, Phe125, Ser126, Arg127, Gly128, Gln129, Met130, Gln131, Lys132, Pro133, Phe134, Glu135, Thr152, Asp153, Ser154, and His157 according to Table III.

51. A method for identifying a modulator of a molecule comprising a PIN1 PPIase substrate-binding site, comprising the steps of:
(a) constructing a computer model of said binding pocket defined by a set of structure coordinates comprising structure coordinates of PIN1 PPIase substrate-binding site amino acids His59, Leu61, Lys63, Ser67, Arg68, Arg69, Cys113, Leu122, Met130, Gln131, Phe134, Glu135, Thr152, Ser154, and His157 according to Table III;
(b) selecting a compound to be evaluated as a potential modulator by a method selected from the group consisting of (i) assembling molecular fragments into said compound, (ii) selecting a compound from a small molecule database, (iii) de novo ligand design of said compound, and (iv) modifying a known inhibitor, or a portion thereof, of a protein kinase;
(c) employing computational means to perform a fitting program operation between computer models of said compound to be evaluated and said binding pocket in order to provide an energy-minimized configuration of said compound in the binding pocket;
(d) evaluating the results of said fitting operation to quantify the association between said compound and the binding pocket model, thereby evaluating the ability of said compound to associate with said binding pocket;
(e) synthesizing said compound; and
(f) contacting said compound with said molecule to determine the ability of said compound to modulate the peptidyl-isomerase activity of said molecule.

52. The method according to claim 51, wherein a set of structure coordinates comprises structure coordinates of PIN1 PPIase substrate-binding amino acids Arg54, Arg56, His59, Leu61, Lys63, Ser67, Arg68, Arg69, Ser72, Trp73, Ser111, Asp112, Cys113, Ser114, Ser115, Ala116, Lys117, Ala118, Arg119, Gly120, Asp121, Leu122, Gly123, Ala124, Phe125, Ser126, Arg127, Gly128, Gln129, Met130, Gln131, Lys132, Pro133, Phe134, Glu135, Thr152, Asp153, Ser154, and His157 according to Table III are used to generate said three-dimensional structure of the molecule comprising a PIN1 PPIase-like binding pocket.

53. A method for screening compounds for PIN1 PPIase modulating activity comprising the steps of:
(a) providing an assay buffer containing a Pintide-PIN1 PPIase polypeptide complex;
(b) adding a test compound; and
(c) measuring the disruption of the Pintide-PIN1 PPIase complex.

54. A method according to claim 53, wherein said method is done in a high-throughput format.

55. A method according to claim 53, wherein said Pintide is labeled with fluorescein.

56. A method according to claim 55, wherein said disruption of the Pintide-PIN1 complex is measured using fluorescence-polarization.


SEQUENCE LISTING (11 sequences)

SEQ ID NO: 1 - DNA, 423 bp, Artificial
  PPlase
atgggcagcagccatcatcatcatcatcacagcagcggcctggtgccgcgcggcagccatatgggcaaaaacgggcagggggagcctgccagggtccgctgctcgcacctgctggtgaagcacagccagtcacggcggccctcgtcctggcggcaggagaagatcacccggaccaaggaggaggccctggagctgatcaacggctacatccagaagatcaagtcgggagaggaggactttgagtctctggcctcacagttcagcgactgcagctcagccaaggccaggggagacctgggtgccttcagcagaggtcagatgcagaagccatttgaagacgcctcgtttgcgctgcggacgggggagatgagcgggcccgtgttcacggattccggcatccacatcatcctccgcactgagtga

SEQ ID NO: 2 - protein, 123 aa, Artificial
  PPlase
GSHMGKNGQGEPARVRCSHLLVKHSQSRRPSSWRQEKITRTKEEALELINGYIQKIKSGEEDFESLASQFSDCSSAKARGDLGAFSRGQMQKPFEDASFALRTGEMSGPVFTDSGIHIILRTE

SEQ ID NO: 3 - DNA, 422 bp, Artificial
  PPlase
atgggcagcagccatcatcatcatcatcacagcagcggcctggtgccgcgcggcagccatatggcaaaaacgggcagggggagcctgccagggtccgctgctcgcacctgctggtgaagcacagccagtcacggcggccctcgtcctggcggcaggagcagatcacccggacccaggaggaggccctggagctgatcaacggctacatccagaagatcaagtcgggagaggaggactttgagtctctggcctcacagttcagcgactgcagctcagccaaggccaggggagacctgggtgccttcagcagaggtcagatgcagaagccatttgaagacgcctcgtttgcgctgcggacgggggagatgagcgggcccgtgttcacggattccggcatccacatcatcctccgcactgagtga

SEQ ID NO: 4 - protein, 123 aa, Artificial
  PPlase
GSHMGKNGQGEPARVRCSHLLVKHSQSRRPSSWRQEQITRTQEEALELINGYIQKIKSGEEDFESLASQFSDCSSAKARGDLGAFSRGQMQKPFEDASFALRTGEMSGPVFTDSGIHIILRTE

SEQ ID NO: 5 - DNA, 36 bp, Artificial
  Primer
agcagccatatgggcaaaaacgggcagggggagcct

SEQ ID NO: 6 - DNA, 30 bp, Artificial
  Primer
cttggatcctcactcagtgcggaggatgat

SEQ ID NO: 7 - protein, 119 aa, Artificial
  PPlase domain
GKNGQGEPARVRCSHLLVKHSQSRRPSSWRQEKITRTKEEALELINGYIQKIKSGEEDFESLASQFSDCSSAKARGDLGAFSRGQMQKPFEDASFALRTGEMSGPVFTDSGIHIILRTE

SEQ ID NO: 8 - DNA, 44 bp, Artificial
  Primer
gcggcaggagcagatcacccggacccaggaggaggccctggagc

SEQ ID NO: 9 - DNA, 44 bp, Artificial
  Primer
gctccagggcctcctcctgggtccgggtgatctgctcctgccgc

SEQ ID NO: 10 - protein, 119 aa, Artificial
  PPlase domain
GKNGQGEPARVRCSHLLVKHSQSRRPSSWRQEQITRTQEEALELINGYIQKIKSGEEDFESLASQFSDCSSAKARGDLGAFSRGQMQKPFEDASFALRTGEMSGPVFTDSGIHIILRTE

SEQ ID NO: 11 - protein, 11 aa, Artificial
  Pintide where the serine is a phosphorylated
FLWFYPSPFLE